(19) United States
(12) Patent Application Publication
(10) Pub. No.: US 2024/0060049 A1
(43) Pub. Date: Feb. 22, 2024

(54) LYOPHILIZED MESENCHYMAL STEM CELLS

(71) Applicant: Habil F KHORAKIWALA, Mumbai, Maharashtra (IN)

(72) Inventors: Habil F KHORAKIWALA, Mumbai, Maharashtra (IN); Vijay SHARMA, Mumbai, Maharashtra (IN); Zahabiya KHORAKIWALA, Mumbai, Maharashtra (IN)

(73) Assignee: Habil F KHORAKIWALA, Mumbai, Maharashtra (IN)

(21) Appl. No.: 18/258,207

(22) PCT Filed: Dec. 19, 2021
(86) PCT No.: PCT/IB2021/061997

§ 371 Date: Jun. 17, 2023

(30) Foreign Application Priority Data
Dec. 19, 2020 (IN) 202021055334

(51) Int. Cl. C12N 5/0775; A01N 1/02

(52) U.S. Cl. CPC C12N 5/0667; A01N 1/0221

(57) ABSTRACT

The present disclosure belongs to the field of stem cell technology. Specifically, the present disclosure relates to a lyophilized powder of mesenchymal stem cells. More specifically, the present disclosure relates to a lyophilized adipose tissue derived mesenchymal stem cells. The present disclosure also relates to the advantageous use of lyophilized mesenchymal stem cells for long-term preservation, easy transportation and distribution of samples in a cost-effective way.

### RELATED PATENT APPLICATIONS

This application claims priority to and benefit of the Indian Patent Application No. 202021055334 filed on Dec. 19, 2020, the disclosures of which are incorporated herein by reference in its entirety as if fully rewritten herein.

### FIELD OF THE INVENTION

The present disclosure relates to the field of stem cell research. Specifically, the disclosure relates to a lyophilized powder of mesenchymal stem cells. More specifically, the disclosure relates to a lyophilized adipose tissue derived mesenchymal stem cells. The present disclosure also relates to the advantageous use of lyophilized mesenchymal stem cells for long-term preservation, easy transportation and distribution of samples in a cost-effective way.

### BACKGROUND OF THE INVENTION

Nowadays, the demand for organ transplantation has been rising rapidly due to the increasing incidence of chronic diseases (e.g., liver cirrhosis and myocardial ischemia), which lead to the end stage failure of many vital organs (e.g., liver and heart). The supply of organs from deceased donors has remained low and insufficient to meet the increasing demand. So, shortage of organs for transplantation has become a major crisis worldwide. To solve the organ shortage problem, regenerative medicine which emphasizes on the use of human stem cells in the treatment, has evolved rapidly.

Stem cells are ultimate candidates for many biomedical applications, particularly cell-based therapies and regenerative medicine. Stem cells are divided into two broad types: embryonic stem cells (ESCs), obtained from the inner cell mass of blastocysts, and adult stem cells, particularly Mesenchymal stem cells (MSCs), found in adult tissues. MSCs hold many advantages over embryonic stem cells (ESCs) and other somatic cells in clinical applications. MSCs are multipotent cells with strong immunosuppressive properties. They can be harvested from various locations in the human body (e.g., bone marrow and adipose tissues).

Adipose tissue as a stem cell source is universally available and has several advantages compared to other sources. It is easily accessible in large quantities with minimal invasive harvesting procedure, and isolation of adipose-derived mesenchymal stromal/stem cells (ASCs) yields a high amount of stem cells, which is essential for stem-cell-based therapies and tissue engineering. Several studies have provided evidence that ASCs in situ reside in a perivascular niche, whereas the exact localization of ASCs in native adipose tissue is still under debate. ASCs are isolated by their capacity to adhere to plastic. Nevertheless, recent isolation and culture techniques lack standardization.

Human adipose-derived stem cells (hASCs) currently represent a viable source of mesenchymal-like stem cells, with similar properties and differentiation potential to bone-marrow-derived mesenchymal stem cells (BM-MSCs) but with a different and more accessible source—the adipose tissue. hASCs are able to produce almost all of the factors that contribute to normal wound healing, and therefore, they are preferred for all types of tissue engineering (TE) and regenerative medical applications. Human adipose-derived stem cells (hASCs) are currently recognized as an attractive and efficient adult stem cell type for regenerative medicine. Still, there are problems that need to be clarified including the mechanisms of the interactions among hASCs and their long-term safety. Only a small number of clinical trials have been performed by now. The majority of clinical trials involving hASCs or hASCs-enriched fat grafts are initial phase clinical trials (phase I or II), while only one trial reached phase IV in human subjects (NCT00616135).

Murphy, M. B. et. al (Exp. Mol. Med. 2013, 45-54) and Fierabracci, A et. al (Curr. Med. Chem. 2016, 23, 3014-3024) describes Mesenchymal stem/stromal cells (MSCs) as an effective tool for the treatment of various diseases, due to their tissue protective and reparative mechanisms. Galipeau, J et. al (Cell Stem Cell 2018, 22, 824-833) has captured the MSC therapeutic effectiveness which has been proved by almost 810 worldwide clinical trials conducted in US until Mar. 31, 2018, with a variety of diseases treated. However, the storage of MSCs is complicated and expensive. The commonly used approach for MSCs storage is cryopreservation using liquid nitrogen.

As of now, cryopreservation represents an efficient method used to preserve and store cells, including hMSCs, for a long-term period. Cryopreservation adopts a principle which utilizes ultralow temperatures (approximately −196° C., e.g., in liquid nitrogen) to halt the metabolic activity of cells while maintaining their life and cell functionality. Cryopreservation is very effective for the pooling of MSCs, to obtain the cell counts required for clinical applications, such as cell-based therapies and regenerative medicine. Upon cryopreservation, it is important to preserve MSCs functional properties including immunomodulatory properties and multilineage differentiation ability. Further, a biosafety evaluation of cryopreserved MSCs is essential prior to their clinical applications. However, the existing cryopreservation methods for MSCs are associated with notable limitations, leading to a need for new or improved methods to be established for a more efficient application of MSCs in stem cell-based therapies.

Elia Bari et. al (Cells 2018, 7, 190) provides a pilot production process for mesenchymal stem/stromal freeze-dried secretome, this was performed in a validated good manufacturing practice (GMP)-compliant cell factory. Secretome was purified from culture supernatants by ultrafiltration, added to cryoprotectant, lyophilized and characterized. They obtained a freeze-dried, “ready-off-the-shelf” and free soluble powder containing extracellular vesicles and proteins. US patent Application No. 2016/0089401A1 relates to cellular compositions and methods relating to the use of aqueous trehalose media to suspend cells.

Overall, recovery of human mesenchymal stem cells and the dehydration potential, allowing them to be shipped coast to coast without special cryoprotective packaging and retaining their growth and function characteristics are the challenges currently being faced in order to preserve MSCs effectively for clinical applications. Solutions aimed at improving this situation are needed.

Bissoyi, A. et. al., in a review article highlighting Recent Advances and Future Directions in Lyophilization and Desiccation of Mesenchymal Stem cells concluded that “[e]nhanced measures of protection are required for successful hydrobiotic engineering of MSCs since existing protocols are not able to ensure robust cell recovery. Although the lyophilisation and desiccation are found to be efficient in some cases, the limitations of individual methods impart certain rigidity of their implementation. At the same time, maintenance of the dried cells viability in long-term storage is another critical issue which needs to be addressed.” (Stem Cells International. Vol. 2016, Article ID 3604203). The review article leaves a challenge to stem cell researchers to demonstrate the lyophilisation of mesenchymal stem cells with considerable attainment of cell viability and other desired advantages such as sample stability at room temperature, defined porous product structure, easy reconstitution by the addition of water or aqueous solution, and easy transportation.

Now it has been surprisingly found by the present inventors that about 15% to about 97% cell viability could be achieved by the lyophilised mesenchymal stem cells according to the present invention. Further, these lyophilised mesenchymal stem cells are suitable for storage at room temperature and would provide the ease of transportation and distribution.

### SUMMARY OF THE INVENTION

The present inventors in view of the background in the area have found a need for the usage of mesenchymal stem cells, by preserving them in a way allowing them to be rapidly available for an application by ensuring high cell viability.

The present inventors while working in the stem cell research area have surprisingly observed about 15% to about 97% cell viability by the lyophilised mesenchymal stem cells according to the present invention. These lyophilised mesenchymal stem cells according to the present invention could be suitable for storage at room temperature and would provide the ease of transportation and distribution.

The present invention also relates to the pharmaceutically acceptable cake that results from lyophilization.

In one embodiment, the present disclosure provides lyophilized MSCs.

In one embodiment, the present disclosure provides a lyophilized powder of mesenchymal stem cells.

In one aspect of an embodiment described herein, the mesenchymal stem cells are selected from the group consisting of umbilical cord mesenchymal stem cells, placental mesenchymal stem cells, adipose tissue derived mesenchymal stem cells, limbal tissue derived mesenchymal stem cells and bone marrow mesenchymal stem cells, and a combination thereof.

In another aspect of the embodiment described herein, the mesenchymal stem cells are adipose tissue derived mesenchymal stem cells.

In another aspect of the embodiment described herein, the mesenchymal stem cells are human mesenchymal stem cells.

In another aspect of the embodiment described herein, the mesenchymal stem cells are human adipose tissue derived mesenchymal stem cells.

In another aspect of the embodiment described herein, the mesenchymal stem cells are exposed to different lyophilisation protocols in presence of various combinations of ingredients.

In another aspect of the embodiment described herein, the mesenchymal stem cells in a lyophilisation mixture comprise various combinations of ingredients.

In another aspect of the embodiment described herein, the mesenchymal stem cells lyophilized powder comprising ingredients, wherein the ingredients are selected from one or more from lyoprotectants, human serum albumin, Glycerol, polyethylene glycol (PEG).

In one aspect of an embodiment described herein, the lyophilized powder of mesenchymal stem cells comprises mesenchymal stem cells and a lyophilisation mixture.

In another aspect of the embodiment described herein, the lyophilization mixture comprises at least one lyoprotectant selected from the group consisting of trehalose, sucrose, lactose, glucose, raffinose, dextran, mannitol, sorbitol, xylitol, and a mixture thereof.

In one aspect of the embodiment described herein, the at least one lyoprotectant is trehalose.

In one aspect of the embodiment described herein, the at least one lyoprotectant is dextran.

In one aspect of the embodiment described herein, the at least one lyoprotectant includes a combination of trehalose and dextran.

In another aspect of an embodiment described herein, the lyophilization mixture comprises human serum albumin.

In yet another aspect of an embodiment described herein, the lyophilization mixture comprises glycerol.

In yet another aspect of an embodiment described herein, the lyophilization mixture comprises poly-ethylene glycol (PEG). In one aspect of the embodiment, the lyophilization mixture comprises PEG 400, PEG 6000, and/or PEG 8000.

In another aspect of the embodiment described herein, the lyophilisation mixture comprises: (a) human serum albumin, and (b) trehalose.

In another aspect of the embodiment described herein, the lyophilisation mixture comprises: (a) human serum albumin, (b) trehalose, and (c) Glycerol.

In another aspect of an embodiment described herein, is the lyophilisation mixture comprises: (a) human serum albumin, (b) trehalose, (c) Glycerol, and (d) Dextran.

In another aspect of an embodiment described herein, is the lyophilisation mixture comprises: (a) human serum albumin, (b) trehalose, (c) Glycerol, and (d) Dextran.

In another aspect of the embodiment described herein, the lyophilisation mixture comprises: (a) human serum albumin, (b) trehalose, (c) Glycerol, (d) Dextran, and (e) polyethylene glycol (PEG).

In another aspect of the embodiment described herein, the lyophilisation mixture comprises: (a) human serum albumin, (b) trehalose, (c) Glycerol, (d) Dextran, and (e) PEG 400.

In another aspect of the embodiment described herein, the lyophilisation mixture comprises: (a) human serum albumin, (b) trehalose, (c) Glycerol, (d) Dextran, and (e) PEG 400, PEG 6000, and/or PEG 8000.

In another aspect of the embodiment described herein, the lyophilisation mixture comprises: (a) human serum albumin, (b) trehalose, (c) PEG 400, and (d) PEG 8000.

In one aspect of the invention, a pharmaceutically acceptable cake resulting from the lyophilization of the mesenchymal stem cells is described.

In another aspect of the invention, the lyophilized mesenchymal stem cells are stored at room temperature.

In another aspect of the invention, the lyophilized mesenchymal stem cells are safe and easy for transportation.

In another aspect of the invention, the lyophilized mesenchymal stem cells are stable after transportation.

In one aspect of the embodiment described herein, the lyophilized powder of mesenchymal cells comprises mesenchymal stem cells and a lyophilization mixture described herein, wherein the viability of mesenchymal stem cells, post lyophilisation, is maintained between about 15% to about 97%.

In one aspect of the embodiment described herein, the lyophilized powder of mesenchymal cells comprises mesenchymal stem cells and a lyophilization mixture described herein, wherein the viability of mesenchymal stem cells, post lyophilisation, is maintained between about 25% to about 90%.

In another aspect of the embodiment described herein, the lyophilized powder of mesenchymal cells comprises mesenchymal stem cells and a lyophilization mixture described herein, wherein the viability of mesenchymal stem cells, post lyophilisation, is reduced or declined to about 0% to about 30%.

In another aspect of the embodiment described herein, the lyophilized mesenchymal stem cells in the lyophilized powder are capable of long-term preservation. Further the lyophilized powder provides an additional advantage of easy transportation and distribution of samples in a cost-effective way.

In yet another aspect of the embodiment described herein, a pharmaceutically acceptable cake of lyophilized mesenchymal stem cells.

In another aspect of the embodiment described herein, the pharmaceutically acceptable cake of lyophilized mesenchymal stem cells, wherein the mesenchymal stem cells are human adipose tissue derived mesenchymal stem cells.

In another aspect of the embodiment described herein, the pharmaceutically acceptable cake of lyophilized mesenchymal stem cells can be solid, powder or granular material.

In another aspect of the embodiment described herein, the pharmaceutically acceptable cake of lyophilized mesenchymal stem cells contain up to five percent water by weight of the cake.

In another aspect of the embodiment described herein, the pharmaceutically acceptable cake of lyophilized mesenchymal stem cells, wherein the mesenchymal stem cells are exposed to different lyophilisation protocols in presence of various combinations of ingredients.

In another aspect of the embodiment described herein, the pharmaceutically acceptable cake of lyophilized mesenchymal stem cells, wherein the mesenchymal stem cells in a lyophilisation mixture comprises various combinations of ingredients.

In another aspect of the embodiment described herein, the pharmaceutically acceptable cake of lyophilized mesenchymal stem cells, wherein the ingredients are selected from one or more from lyoprotectants, human serum albumin, Glycerol, polyethylene glycol (PEG).

In another aspect of the embodiment described herein, the pharmaceutically acceptable cake of lyophilized mesenchymal stem cells comprising ingredients, wherein the ingredient is human serum albumin.

In another aspect of the embodiment described herein, the ingredients, wherein the lyoprotectants are selected from one or a mixture of several of trehalose, sucrose, lactose, glucose, raffinose, dextran, mannitol, sorbitol or xylitol.

In another aspect of the embodiment described herein, the pharmaceutically acceptable cake of lyophilized mesenchymal stem cells, wherein the mesenchymal stem cells viability post lyophilisation is between about 25% to about 90%.

In one embodiment described herein, a composition comprising lyophilized MSCs is provided. In one aspect of the embodiment described herein, a composition comprising lyophilized powder of MSCs is provided.

In another embodiment described herein, a pharmaceutical composition comprising lyophilized MSCs is provided. In one aspect of the embodiment described herein, a pharmaceutical composition comprising lyophilized powder of MSCs is provided. The pharmaceutical compositions described herein may also contain one or more anti-caking agents known to one of ordinary skill in the art.

In one embodiment disclosed herein, a kit comprising the lyophilized MSCs is provided. In one aspect of the embodiment disclosed herein, a kit comprising lyophilized powder of MSCs is provided. In another aspect of the embodiment disclosed herein, a kit comprising a pharmaceutical composition comprising lyophilized MSCs is provided. In yet another aspect of the embodiment disclosed herein, a kit comprising a pharmaceutical composition comprising lyophilized powder of MSCs is provided.

In one aspect of the embodiment described herein, the mesenchymal stem cells, post lyophylization, express positive markers. In one aspect of the embodiment, the positive markers comprise one or more selected from the group consisting of CD90, CD105, CD73, CD44, CD29, CD13, CD166, CD10, CD49e and CD59.

In one aspect of the embodiment described herein, the mesenchymal stem cells, post lyophylization, do not express negative markers. In one aspect of the embodiment, the negative markers comprise one or more selected from the group consisting of CD34, CD45, CD14, CD11 b, CD19, CD56 and CD146.

These and other aspects of the embodiments herein will be better appreciated and understood when considered in conjunction with the following description. It should be understood, however, that the following description, while indicating preferred embodiments and numerous specific details thereof, are given by way of illustration and not of limitation. Many changes and modifications may be made within the scope of the embodiments herein without departing from the spirit thereof, and the embodiments described herein include all such modifications.

### BRIEF DESCRIPTION OF THE FIGURES

FIG. 1 shows representative images of lyophilized cakes of combinations 4A to 4G.

FIG. 2 shows representative images of lyophilized cakes of combinations 5A to 5X.

FIG. 3 shows representative images of lyophilized cakes of combinations 6A to 6Z.

FIG. 4 shows representative images of lyophilized cakes of combinations 7A to 7X.

FIG. 5 shows representative images of lyophilized cakes of combinations 8A to 8M.

FIG. 6 shows representative images of lyophilized cakes of combinations 9A to 9K.

FIG. 7 shows representative images of lyophilized cakes of combinations 10A and 10B.

FIG. 8 shows representative images of lyophilized cakes of combinations 11A to 11V.

FIG. 9 shows representative images of lyophilized cakes of combinations 12A to 12F.

### DETAILED DESCRIPTION OF THE INVENTION

As mentioned above, there remains a need in the overall recovery of human mesenchymal stem cells and the dehydration potential, allowing them to be shipped coast to coast without special cryoprotective packaging and retaining their growth and function characteristics in order to preserve MSCs effectively for clinical applications.

The inventors have now surprisingly found that a lyophilized powder of mesenchymal stem cells as described herein, maintained a viability of mesenchymal stem cells, post lyophilisation, from about 15% to about 97%. It was further surprising that these lyophilised mesenchymal stem cells were suitable for storage at room temperature. In addition, it was surprising to find that the lyophylized mesenchymal stem cells were suitable for storage at 2° C. to 8° C.

The embodiments described herein, and the various features and advantageous details thereof, are explained more fully with reference to the non-limiting embodiments that are detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Unless otherwise indicated, all numbers expressing quantities of ingredients, properties such as molecular weight, reaction conditions, and so forth used in this disclosure and the appended claims are to be understood as being modified in all instances by the term “about.” Accordingly, unless indicated to the contrary, the numerical parameters set forth in this disclosure and the appended claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the embodiments are approximations, the numerical values set forth in the specific examples are reported as precisely as possible. Any numerical value, however, inherently contains certain errors necessarily resulting from the standard deviation found in their respective testing measurements.

Ranges may be expressed herein as from “about” one particular value, and/or to “about” another particular value. When such a range is expressed, also specifically contemplated and considered disclosed is the range-1 from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Reference will now be made to the exemplary embodiments, and specific language will be used herein to describe the same. It should nevertheless be understood that no limitation of the scope of the invention is thereby intended. Alterations and further modifications of the inventive features illustrated herein, and additional applications of the principles of the invention as illustrated herein, which would occur to one of ordinary skills in the relevant art and having possession of this disclosure, are to be considered within the scope of the invention. It must be noted that, as used in this specification and the appended claims, the singular forms “a”, “an”, and “the” include plural referents unless the content clearly dictates otherwise. All references including patents, patent applications, and literature cited in the specification are expressly incorporated herein by reference in their entirety. The term “medication” as used herein refers to a medicine or pharmaceutical drug, or simply drug; which is used to diagnose, cure, treat, or prevent disease. The term “medication” can also be refereed as the administration of a drug or medicine.

The term “confluency” as used herein refers to the area that each cell occupies the viable cells per ml, the ratio of area occupied by the cells and the total area available, the area below the line of a growth curve. In cell culture biology, confluency is the term commonly used as a measure of the number of the cells in a cell culture dish or a flask and refers to the coverage of the dish or the flask by the cells. For example, 100 percent confluency means the dish is completely covered by the cells, and therefore no more room left for the cells to grow; whereas 50 percent confluency means roughly half of the dish is covered and there is still room for cells to grow.

The term “cell viability” as used herein refers to a measure of the proportion of live, healthy cells within a population. Typically, cell viability assays provide readout of cell health through measurement of metabolic activity, ATP content, or cell proliferation. A viability assay is an assay that is created to determine the ability of organs, cells or tissues to maintain or recover a state of survival. Viability can be distinguished from the all-or-nothing states of life and death by the use of a quantifiable index that ranges between the integers of 0 and 1 or, if more easily understood, the range of 0% and 100%. Viability can be observed through the physical properties of cells, tissues, and organs. Some of these include mechanical activity, motility, such as with spermatozoa and granulocytes, the contraction of muscle tissue or cells, mitotic activity in cellular functions, and more. Viability assays provide a more precise basis for measurement of an organism's level of vitality.

Viability assays can lead to more findings than the difference of living versus non-living. According to one embodiment of the present disclosure, a viability assay can be used to assess the success of Lyophilisation.

The term “Flow cytometry” as used herein refers to a technique used to detect and measure physical and chemical characteristics of a population of cells or particles. In this process, a sample containing cells or particles is suspended in a fluid and injected into the flow cytometer instrument. Flow cytometry analyses individual cells, thereby permitting the determination of sample heterogeneity. As viability is ultimately a characteristic of an individual cell, an approach such as this is essential for meaningful results to be obtained. Flow cytometric analysis at the single-cell level allows distributions of multiple cell properties to be determined, allowing identifications of subpopulations of cells that may be characterized on a spectrum from “maximum viability” through to death and, potentially, degradation.

MSCs are typically identified by their co-expression of CD73, CD90, and CD105. To demonstrate an alternative method for MSC detection, expanded MSC are generally screened for MSC markers CD73, CD90 and CD105. The MSC markers CD73, CD90 and CD105 were detected by flow cytometry. Flow cytometry provides a rapid and reliable method to quantify viable cells in a cell suspension. Determination of cell viability is critical when evaluating the physiological state of cells, such as in response to cytotoxic drugs and environmental factors, or during the progression of cancer and other disease states. In addition, it is often necessary to detect dead cells in a cell suspension in order to exclude them from analysis. Dead cells can generate artifacts as a result of non-specific antibody binding or unwanted uptake of fluorescent probes. One method to identify the two cell populations is by dye exclusion. Live cells have intact membranes that exclude a variety of dyes that easily penetrate the damaged, permeable membranes of non-viable cells. Several different fluorochromes can be used to stain non-viable cells including 7-amino actinomycin D (7-AAD). 7-AAD is a membrane impermeant dye that is generally excluded from viable cells. It binds to double stranded DNA by intercalating between base pairs in G-C-rich regions. 7-AAD can be excited at 488 nm with an argon laser. It has a relatively large Stokes shift, emitting at a maximum wavelength of 647 nm. Because of these spectral characteristics, 7-AAD can be used in combination with other fluorochromes excited at 488 nm such as fluorescein isothiocyanate (FITC) and phycoerythrin (PE).

The term “Lyophilization or freeze-drying” refers to a process used to freeze materials and then remove the frozen water by sublimation; that means ice turns directly into vapour leaving out the liquid phase. In general, the freeze-drying or lyophilization technique is to dissolve, suspend, or emulsify a compound or formulation; freeze the resultant solution, suspension, or emulsion; and then to apply a vacuum thereto to sublimate/evaporate the solvents and other liquids in the frozen mass used to dissolve, suspend or emulsify the material. Lyophilization/freeze-drying is most often used method for gentle preservation certain substances, such as temperature sensitive Food or especially medication. Here the substances dried in the frozen state and can be added of water or another solvent especially easily return to its original state. With this, the processes are generally based on the starting product's temperatures frozen down to −70° C. The process of drying, in pressure-resistant containers (Lyophilizers), takes place under high vacuum wherein the water through Sublimation withdrawn, and the freeze-dried substance is obtained.

The pharmaceutically acceptable cake can be administered orally or parenterally after reconstitution or swallowed orally without reconstitution. As used herein, a “pharmaceutically acceptable cake” refers to a non-collapsed solid drug product remaining after lyophilization that has certain desirable characteristics, e.g., pharmaceutically acceptable, long-term stability, a short reconstitution time, an elegant appearance and maintenance of the characteristics of the original Solution upon reconstitution. The pharmaceutically acceptable cake can be solid, powder or granular material. As used herein a “lyophilized powder of mesenchymal stem cells” can also refer to pharmaceutically acceptable cake of lyophilized mesenchymal stem cells. The pharmaceutically acceptable cake may also contain up to five percent water by weight of the cake.

The term “ingredients” used in the present invention refers to pharmaceutical excipients routinely used in medicinal products. Examples of ingredients or excipients include antioxidants, buffers, chelating agents and lyoprotectants. Examples of lyoprotectants include sugars, PEG and certain inorganic salts. Examples of polymers include polyvinyl pyrrolidine (PVP), polyethylene glycol (PEG) and polyvinyl alcohol (PVA). The most preferred ingredients according to present invention are selected from one or more of lyoprotectants, human serum albumin, Glycerol, polyethylene glycol (PEG) or polyvinyl pyrrolidine (PVP).

The term “lyoprotectant” refers to a substance that is added to a formulation in order to protect the active ingredients (for example, mesenchymal stem cells in the instant case). It is a substance added to something undergoing lyophilization in order to prevent damage. Lyoprotectans generally are the compounds which are used in lyophilisation to protect the products that are sensitive to occurring dehydration. Lyoprotectants routinely includes sugars, polyalcohols, and their derivatives. In a preferred embodiment, lyoprotectants is at least one sugar selected from the group consisting of trehalose, sucrose, lactose, glucose, raffinose, dextran, mannitol, sorbitol, xylitol, and a combination thereof.

Human serum albumin is the primary protein present in human blood plasma. The main function of albumin is to maintain the oncotic pressure of blood. It binds to water, cations (such as Ca2+, Na+ and K+), fatty acids, hormones, bilirubin, thyroxine (T4) and pharmaceuticals (including barbiturates). Albumin represents approximately 50% of the total protein content in healthy humans. Human albumin is a small globular protein (molecular weight: 66.5 kDa), consisting of a single chain of 585 amino acids organized in three repeated homolog domains (sites I, II, and III). Each domain comprises two separate sub-domains (A and B).

Human serum albumin (HSA) typically referred as soluble, globular, and unglycosylated monomeric protein; it functions primarily as a carrier protein for steroids, fatty acids, and thyroid hormones, and plays an important role in stabilizing extracellular fluid volume. HSA is widely used clinically to treat serious burn injuries, hemorrhagic shock, hypoproteinemia, fetal erythroblastosis, and ascites caused by cirrhosis of the liver. HSA is also used as an excipient for vaccines or therapeutic protein drugs and as a cell culture medium supplement in the production of vaccines and pharmaceuticals.

Trehalose, also known as mycose or tremalose, is an alpha-linked disaccharide formed by an a,a-1,1-glucoside bond between two a-glucose units. It has a chemical name of (2R,3S,4S,5R,6R)-2-(hydroxymethyl)-6-[(2R,3R,4S,5S, 6R)-3,4,5-trihydroxy-6-(hydroxymethyl)oxan-2-yl]oxyoxane-3,4,5-triol (IUPAC naming convention).

Dextran has frequently been used as a polysaccharide lyoprotectant in dry protein formulations, mainly due to its high glass transition temperature, which enables room temperature storage. As an inert additive, dextran is particularly suitable to be used as a preservative in pharmaceutical products. As a result, there have been numerous drugs in the market that contain dextran as a preservative, including biologics. Dextran provides an excellent amorphous bulking agent, which can be lyophilized rapidly with formation of strong, elegant cake structure. Dextran when used along with sucrose or trehalose during lyophilization results into improved storage stability.

Glycerol is a triol with a structure of propane substituted at positions 1, 2 and 3 by hydroxy groups. It has a role as an osmolyte, a solvent, a detergent, a human metabolite, an algal metabolite, a Saccharomyces cerevisiae metabolite, an Escherichia coli metabolite and a mouse metabolite. It is an alditol and a triol.

Polyethylene glycols (PEGs) are products made of condensed ethylene oxide and water that can contain various derivatives and have various functions. Because many PEG types are hydrophilic, they are favourably used as enhancers of penetration, and used heavily in topical dermatological preparations. PEGs, along with their many non-ionic derivatives, are widely utilized in cosmetic products as surfactants, emulsifiers, cleansing agents, humectants, and skin conditioners.

Polyethylene glycol 400 (PEG 400) is a low-molecular-weight grade of polyethylene glycol with a low-level toxicity. It is very hydrophilic, which renders it a useful ingredient in drug formulations to augment the solubility and bioavailability of weakly water-soluble drugs. It is used in ophthalmic solutions for the relief of burning, irritation and/or discomfort that follows dryness of the eye. PEG “400” indicates that the average molecular weight of the specific PEG is 400.

Polyethylene glycol 8000 (PEG 8000) is a high molecular polyethylene glycol (macrogol) mainly used as solvent for various preparations. The high molecular weight PEG is soluble in water and organic solvents such as alcohols. It can be blended with other PEG molecular weights to achieve the desired properties, i.e. viscosity.

“Trypsinization” is the process of cell dissociation using trypsin, a proteolytic enzyme which breaks down proteins, to dissociate adherent cells from the vessel in which they are being cultured. When added to a cell culture, trypsin breaks down the proteins which enable the cells to adhere to the vessel.

The passage number of a cell culture is a record of the number of times the culture has been subcultured, i.e. harvested and reseeded into multiple ‘daughter’ cell culture flasks. When cells are trypsinized for freezing and then thawed and reseeded, this represents one passage, albeit with time out in the freezer.

“Room temperature” as used herein refers to normal storage conditions, which means storage in a dry, clean, well-ventilated area at room temperatures between −25° C. to 30° C. or up to 45° C., depending on climatic conditions. “Room temperature” can also refer to a temperature prevailing in a work area.

As used herein a “pharmaceutical composition” refers to a therapeutically effective amount of the lyophilized Mesenchymal Stem Cells (MSCs) or lyophilized powder of MSCs as described herein. The pharmaceutical composition may be in combination with other components such as pharmaceutically acceptable carriers, which may facilitate administration of the lyophilized Mesenchymal Stem Cells (MSCs) or lyophilized powder of MSCs to a subject in need thereof.

The term “pharmaceutically acceptable carrier” refers to a carrier or a diluent that does not cause significant irritation to a subject and does not abrogate the biological activity and properties of the lyophilized Mesenchymal Stem Cells (MSCs) or lyophilized powder of MSCs. A pharmaceutically acceptable carrier may include, but is not limited to, physiological saline, ringers, phosphate buffered saline, and other carriers known in the art.

Following are the aspects of the present disclosure.

In one embodiment, the present disclosure provides lyophilized mesenchymal stem cells (MSCs).

In one embodiment, the present disclosure provides a lyophilized powder of mesenchymal stem cells.

In one aspect of an embodiment described herein, the mesenchymal stem cells are selected from the group consisting of umbilical cord mesenchymal stem cells, placental mesenchymal stem cells, adipose tissue derived mesenchymal stem cells, limbal tissue derived mesenchymal stem cells and bone marrow mesenchymal stem cells, and a combination thereof.

In another aspect of the embodiment described herein, the mesenchymal stem cells are adipose tissue derived mesenchymal stem cells.

In another aspect of the embodiment described herein, the mesenchymal stem cells are human mesenchymal stem cells.

In another aspect of the embodiment described herein, the mesenchymal stem cells are human adipose tissue derived mesenchymal stem cells.

In another aspect of the embodiment described herein, the mesenchymal stem cells are exposed to different lyophilisation protocols in presence of various combinations of ingredients.

In another aspect of the embodiment described herein, the mesenchymal stem cells in a lyophilisation mixture comprise various combinations of ingredients.

In another aspect of the embodiment described herein, the mesenchymal stem cells lyophilized powder comprising ingredients, wherein the ingredients are selected from one or more from lyoprotectants, human serum albumin, Glycerol, polyethylene glycol (PEG).

In one aspect of an embodiment described herein, the lyophilized powder of mesenchymal stem cells comprises mesenchymal stem cells and a lyophilisation mixture.

In another aspect of the embodiment described herein, the lyophilization mixture comprises at least one lyoprotectant selected from the group consisting of trehalose, sucrose, lactose, glucose, raffinose, dextran, mannitol, sorbitol, xylitol, and a mixture thereof.

In one aspect of the embodiment described herein, the at least one lyoprotectant is trehalose.

In one aspect of the embodiment described herein, the at least one lyoprotectant is dextran.

In one aspect of the embodiment described herein, the at least one lyoprotectant includes a combination of trehalose and dextran.

In another aspect of an embodiment described herein, the lyophilization mixture comprises human serum albumin.

In yet another aspect of an embodiment described herein, the lyophilization mixture comprises glycerol.

In yet another aspect of an embodiment described herein, the lyophilization mixture comprises poly-ethylene glycol (PEG). In one aspect of the embodiment, the lyophilization mixture comprises PEG 400 and/or PEG 8000.

In another aspect of the embodiment described herein, the lyophilisation mixture comprises: (a) human serum albumin, and (b) trehalose.

In another aspect of the embodiment described herein, the lyophilisation mixture comprises: (a) human serum albumin, (b) trehalose, and (c) Glycerol.

In another aspect of an embodiment described herein, is the lyophilisation mixture comprises: (a) human serum albumin, (b) trehalose, (c) Glycerol, and (d) Dextran.

In another aspect of the embodiment described herein, the lyophilisation mixture comprises: (a) human serum albumin, (b) trehalose, (c) Glycerol, (d) Dextran, and (e) polyethylene glycol (PEG).

In another aspect of the embodiment described herein, the lyophilisation mixture comprises: (a) human serum albumin, (b) trehalose, (c) Glycerol, (d) Dextran, and (e) PEG 400.

In another aspect of the embodiment described herein, the lyophilisation mixture comprises: (a) human serum albumin, (b) trehalose, (c) PEG 400, and (d) PEG 8000.

In one aspect of the invention, a pharmaceutically acceptable cake resulting from the lyophilization of the mesenchymal stem cells is described.

In another aspect of the invention, the lyophilized mesenchymal stem cells are stored at room temperature.

In another aspect of the invention, the lyophilized mesenchymal stem cells are safe and easy for transportation.

In another aspect of the invention, the lyophilized mesenchymal stem cells are stable after transportation.

In one aspect of the embodiment described herein, the lyophilized powder of mesenchymal cells comprises mesenchymal stem cells and a lyophilization mixture described herein, wherein the viability of mesenchymal stem cells, post lyophilisation, is maintained between about 15% to about 97%.

In one aspect of the embodiment described herein, the lyophilized powder of mesenchymal cells comprises mesenchymal stem cells and a lyophilization mixture described herein, wherein the viability of mesenchymal stem cells, post lyophilisation, is maintained between about 25% to about 90%.

In another aspect of the embodiment described herein, the lyophilized powder of mesenchymal cells comprises mesenchymal stem cells and a lyophilization mixture described herein, wherein the viability of mesenchymal stem cells, post lyophilisation, is reduced or declined to about 0% to about 30%.

In another aspect of the embodiment described herein, the lyophilized mesenchymal stem cells in the lyophilized powder are capable of long-term preservation. Further the lyophilized powder provides an additional advantage of easy transportation and distribution of samples in a cost-effective way.

In yet another aspect of the embodiment described herein, a pharmaceutically acceptable cake of lyophilized mesenchymal stem cells.

In another aspect of the embodiment described herein, the pharmaceutically acceptable cake of lyophilized mesenchymal stem cells, wherein the mesenchymal stem cells are human adipose tissue derived mesenchymal stem cells.

In another aspect of the embodiment described herein, the pharmaceutically acceptable cake of lyophilized mesenchymal stem cells can be solid, powder or granular material.

In another aspect of the embodiment described herein, the pharmaceutically acceptable cake of lyophilized mesenchymal stem cells contain up to five percent water by weight of the cake.

In another aspect of the embodiment described herein, the pharmaceutically acceptable cake of lyophilized mesenchymal stem cells, wherein the mesenchymal stem cells are exposed to different lyophilisation protocols in presence of various combinations of ingredients.

In another aspect of the embodiment described herein, the pharmaceutically acceptable cake of lyophilized mesenchymal stem cells, wherein the mesenchymal stem cells in a lyophilisation mixture comprises various combinations of ingredients.

In another aspect of the embodiment described herein, the pharmaceutically acceptable cake of lyophilized mesenchymal stem cells, wherein the ingredients are selected from one or more from lyoprotectants, human serum albumin, Glycerol, polyethylene glycol (PEG).

In another aspect of the embodiment described herein, the pharmaceutically acceptable cake of lyophilized mesenchymal stem cells comprising ingredients, wherein the ingredient is human serum albumin.

In another aspect of the embodiment described herein, the ingredients, wherein the lyoprotectants are selected from one or a mixture of several of trehalose, sucrose, lactose, glucose, raffinose, dextran, mannitol, sorbitol or xylitol.

In another aspect of the embodiment described herein, the pharmaceutically acceptable cake of lyophilized mesenchymal stem cells, wherein the mesenchymal stem cells viability post lyophilisation is between about 15% to about 97%.

The instant disclosure provides a lyophylized powder of mesenchymal stem cells comprising mesenchymal stem cells and a lyophylization mixture, wherein the viability of mesenchymal stem cells, post lyophylization, is maintained between about 15% to about 97%. In one embodiment, the viability of mesenchymal stem cells, post lyophylization, is maintained at about 15%, about 16%, about 17%, about 18%, about 19%, about 20%, about 21%, about 22%, about 23%, about 24%, at about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, or about 97%.

In another aspect of the embodiment described herein, the pharmaceutically acceptable cake of lyophilized mesenchymal stem cells, wherein the mesenchymal stem cells viability post lyophilisation is between about 25% to about 90%.

The instant disclosure provides a lyophylized powder of mesenchymal stem cells comprising mesenchymal stem cells and a lyophylization mixture, wherein the viability of mesenchymal stem cells, post lyophylization, is maintained between about 25% to about 90%. In one embodiment, the viability of mesenchymal stem cells, post lyophylization, is maintained at about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%.

In one embodiment, the viability of mesenchymal stem cells, post lyophilization, is reduced to about 0% to about 30%. In one aspect of the embodiment, the viability of mesenchymal stem cells, post lyophilization, is reduced to about 0.1%, about 0.5%, about 1%, about 2.5%, about 5%, about 7.5%, about 10%, about 12.5%, about 15%, about 17.5%, about 20%, about 22.5%, about 25%, about 27.5%, about 30%, about 32.5%, about 35%, about 37.5%, or about 40%.

In one embodiment disclosed herein, the mesenchymal stem cells can be selected from the group consisting of umbilical cord mesenchymal stem cells, placental mesenchymal stem cells, adipose tissue derived mesenchymal stem cells, limbal tissue derived mesenchymal stem cells, bone marrow mesenchymal stem cells, and a combination thereof.

In one embodiment disclosed herein, the lyophilization mixture comprises lyoprotectants that include, but not limited to, at least one antioxidant, at least one sugar, at least one membrane stabilizer, at least one high molecular weight molecule. In one aspect of the embodiment the at least one sugar is selected from the group consisting of trehalose, sucrose, lactose, glucose, raffinose, dextran, mannitol, sorbitol, xylitol and a combination thereof.

In one aspect of the embodiment disclosed herein, the at least one sugar is present in an amount of about 25 mM to about 1000 mM. In a preferred embodiment, the at least one sugar is present in amount of about 25 mM, about 50 mM, about 75 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, about 500 mM, about 525 mM, about 550 mM, about 575 mM, about 600 mM, about 625 mM, about 650 mM, about 675 mM, about 700 mM, about 725 mM, about 750 mM, about 775 mM, about 800 mM, about 825 mM, about 850 mM, about 875 mM, about 900 mM, about 925 mM, about 950 mM, about 975 mM, or about 1000 mM. In one embodiment, the at least one sugar is trehalose.

In one aspect of the embodiment disclosed herein, the at least one sugar is present in an amount of about 0.01% (w/w) to about 10% (w/w) of the total lyophilization mixture. In another aspect of the embodiment, the at least one sugar is present in an amount of about 0.01% (w/w), about 0.02% (w/w), about 0.03% (w/w), about 0.04% (w/w), about 0.05% (w/w), about 0.06% (w/w), about 0.07% (w/w), about 0.08% (w/w), about 0.09% (w/w), about 0.1% (w/w), about 0.15% (w/w), about 0.2% (w/w), about 0.25% (w/w), about 0.3% (w/w), about 0.35% (w/w), about 0.4% (w/w), about 0.45% (w/w), about 0.5% (w/w), about 0.6% (w/w), about 0.7% (w/w), about 0.80% (w/w), about 0.9% (w/w), about 10% (w/w), about 1.50% (w/w), about 2% (w/w), about 2.5% (w/w), about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) of the total lyophilization mixture. In one embodiment, the at least one sugar is dextran.

In another aspect of the embodiment disclosed herein, the at least one sugar comprises trehalose in an amount of about 25 mM to about 1000 mM and dextran in an amount of about 0.01% (w/w) to about 5% (w/w) of the total lyophilization mixture.

In one embodiment disclosed herein, the lyophilization mixture comprises human serum albumin (HSA). In another embodiment disclosed herein, the HSA is present in an amount of about 0.01% (w/w) to about 10% (w/w) of the total lyophilization mixture. In another aspect of the embodiment, the HSA is present in an amount of about 0.01% (w/w), about 0.02% (w/w), about 0.03% (w/w), about 0.04% (w/w), about 0.05% (w/w), about 0.06% (w/w), about 0.07% (w/w), about 0.08% (w/w), about 0.09% (w/w), about 0.1% (w/w), about 0.150% (w/w), about 0.2% (w/w), about 0.25% (w/w), about 0.3% (w/w), about 0.355% (w/w), about 0.4% (w/w), about 0.45% (w/w), about 0.5% (w/w), about 0.6% (w/w), about 0.7% (w/w), about 0.8% (w/w), about 0.9% (w/w), about 1% (w/w), about 1.5% (w/w), about 2% (w/w), about 2.5% (w/w), about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) of the total lyophilization mixture.

In one embodiment disclosed herein, the lyophilization mixture comprises one or more polyalcohols, (e.g. glycerol) that are conventionally used in preservation of biological material. In one aspect of the embodiment disclosed herein, the lyophilization mixture comprises glycerol in an amount of about 0.01% (w/w) to about 5% (w/w) of the total lyophilization mixture. In another aspect of the embodiment, the glycerol is present in an amount of about 0.01% (w/w), about 0.02% (w/w), about 0.03% (w/w), about 0.04% (w/w), about 0.05% (w/w), about 0.06% (w/w), about 0.07% (w/w), about 0.08% (w/w), about 0.09% (w/w), about 0.10% (w/w), about 0.150% (w/w), about 0.2% (w/w), about 0.25% (w/w), about 0.3% (w/w), about 0.35% (w/w), about 0.4% (w/w), about 0.45% (w/w), about 0.5% (w/w), about 0.6% (w/w), about 0.7% (w/w), about 0.8% (w/w), about 0.9% (w/w), about 1% (w/w), about 1.5% (w/w), about 2% (w/w), about 2.5% (w/w), about 3% (w/w), about 3.5% (w/w), about 4% (w/w), about 4.5% (w/w), and about 5% (w/w) of the total lyophilization mixture.

In one embodiment disclosed herein, the lyophilization mixture comprises PEG 400. In another embodiment disclosed herein, the PEG 400 is present in an amount of about 0.01% (w/w) to about 10% (w/w) of the total lyophilization mixture. In another aspect of the embodiment, the HSA is present in an amount of about 0.01% (w/w), about 0.02% (w/w), about 0.03% (w/w), about 0.04% (w/w), about 0.05% (w/w), about 0.06% (w/w), about 0.07% (w/w), about 0.08% (w/w), about 0.09% (w/w), about 0.10% (w/w), about 0.150% (w/w), about 0.2% (w/w), about 0.25% (w/w), about 0.3% (w/w), about 0.35% (w/w), about 0.4% (w/w), about 0.45% (w/w), about 0.5% (w/w), about 0.6% (w/w), about 0.7% (w/w), about 0.8% (w/w), about 0.9% (w/w), about 1% (w/w), about 1.5% (w/w), about 2% (w/w), about 2.5% (w/w), about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), or about 10% (w/w) of the total lyophilization mixture.

In one aspect of the embodiment described herein, the mesenchymal stem cells, post lyophylization, express positive markers. In one aspect of the embodiment, the positive markers comprise one or more markers selected from the group consisting of CD90, CD44, CD29, CD105, CD13, CD34, CD73, CD166, CD10, CD49e and CD59. In another aspect of the embodiment described herein, at least about 60% to about 98% of the mesenchymal stem cells, post lyophylization, express one or more markers selected from the group consisting of CD90, CD44, CD29, CD105, CD13, CD34, CD73, CD166, CD10, CD49e and CD59. In yet another aspect of the embodiment described herein, at least about 60%, at least about 65%, at least about 70%, at least about 75%, at least about 80%, at least about 85%, at least about 90%, at least about 95%, at least 96%, at least about 97%, or at least about 98% of the mesenchymal stem cells, post lyophylization, express one or more markers selected from the group consisting of CD90, CD44, CD29, CD105, CD13, CD34, CD73, CD166, CD10, CD49e and CD59.

In one aspect of the embodiment described herein, the mesenchymal stem cells, post lyophylization, express negative markers. In one aspect of the embodiment, the negative markers comprise one or more selected from the group consisting of CD31, CD45, CD14, CD11b, CD19, CD56 and CD146. In another aspect of the embodiment described herein, no more than about 2% to about 20% of the mesenchymal stem cells, post lyophylization, express one or more markers selected from the group consisting of CD31, CD45, CD14, CD11b, CD19, CD56 and CD146. In yet another embodiment, no more than about 2%, no more than about 4%, no more than about 6%, no more than about 8%, no more than about 10%, no more than about 12%, no more than about 14%, no more than about 16%, no more than about 18%, or no more than about 20% of the mesenchymal stem cells, post lyophylization, express one or more markers selected from the group consisting of CD31, CD45, CD14, CD11b, CD19, CD56 and CD146.

In one aspect of an embodiment described herein, the chromosomal, genomic, and epigenomic profiles of the mesenchymal stem cells, post lyophylization, may be evaluated and compared at different passages during in vitro propagation.

In one aspect of an embodiment described herein, after lyophilization, the lyophilized mesenchymal stem cell becomes a cake. Such a cake should be pharmaceutically acceptable. As used herein, a “pharmaceutically acceptable cake” refers to a non-collapsed solid drug product remaining after lyophilization that has certain desirable characteristics, e.g., pharmaceutically acceptable, long-term stability, a short reconstitution time, an elegant appearance and maintenance of the characteristics of the original Solution upon reconstitution. The pharmaceutically acceptable cake can be solid, powder or granular material. The pharmaceutically acceptable cake may also contain up to five percent water by weight of the cake.

While the present invention has been described in terms of its specific embodiments, certain modifications and equivalents will be apparent to those skilled in the art and are intended to be included within the scope of the invention.

### EXAMPLES

The present disclosure will be further described below in conjunction with specific embodiments, and the advantages and characteristics of the present disclosure will become clearer with the description. However, these embodiments are only exemplary and do not constitute any limitation to the scope of the present disclosure. Those skilled in the art should understand that the details and forms of the technical solutions of the present disclosure can be modified or replaced without departing from the spirit and scope of the present disclosure, but these modifications and replacements fall within the protection scope of the present disclosure.

### Example 1

The effect of lyophilization on Mesenchymal Stem Cells (MSCs) was studied by exposing cells to different lyophilization protocols in presence of various combinations of ingredients. The viability of cells was analyzed before and after lyophilization.

TABLE 1
Sample Details

| | Mesenchymal |
|---|---|
| Cells used | Stem Cells |
| Passage No. | P2 |

a. Cell Suspension Preparation

Cells were grown in growth medium (DMEM-LG) to attain 90% confluency. Upon attaining 90% confluence, the cells were exposed to 100 mM Trehalose in DMEM-LG for 24 hours at 37° C. Cells were then trypsinized and re-suspended in 9 different combinations of Lyophilization Solutions. One part of the cell suspension was used to perform pre-lyophilization viability and cell surface marker analysis by flow cytometry.

TABLE 2
Mesenchymal Stem Cells lyophilization
mixture in a combination of ingredients

| Sr. | combinations of ingredients |
|---|---|
| a | 150 mM Trehalose |
| b | 1% HSA |
| c | 0.1% Glycerol |
| d | 0.1% Dextran |
| e | 0.1% PEG400 |
| f | 150 mM Trehalose + 1% HSA |
| g | 150 mM Trehalose + 1% HSA + |
| | 0.1% Glycerol |
| h | 150 mM Trehalose + 1% HSA + |
| | 0.1% Glycerol + 0.1% Dextran |
| i | 150 mM Trehalose + 1% HSA + |
| | 0.1% Glycerol + 0.1% Dextran + |
| | 0.1% PEG400 |
| j | Vehicle Control |

All solutions prepared in DMEM-LG w/o Phenol red, Glucose and L-Glutamine (Vehicle).

TABLE 3
Pre-lyophilization viability and cell
surface marker analysis by flow cytometry

| | |
|---|---|
| Viability by 7AAD | 78.96% |
| CD90 | 65.71% |
| CD73 | 65.18% |
| CD105 | 56.25% |

As can be seen from above table, the observed pre-lyophilization cell viability analysed by 7AAD staining is 78.96%. The cell surface marker analysis shows that 65.71% of the cell population expresses CD90 expression, 65.18% of the cell population express CD73 expression and 56.25% of the cell population expresses CD105 expression.

b. Post-Lyophylization Viability Analysis by Flow Cytometry:

Viability of the lyophilized cells was analyzed by 7AAD staining 17 days post lyophilization. Each combination of the lyophilized product was individually reconstituted in 1×PBS and cells were centrifuged at 300 g to obtain a pellet. The pellet was re-suspended in 1×PBS. The cells were then stained with 7AAD dye to analyze cell viability by flow cytometry.

Each combination of mesenchymal stem cells lyophilization mixtures as per table 2 was lyophilized. After lyophilization, the lyophilized products were sealed with 20 mm aluminium flip of seals and stored at 2-8° C. for a period of minimum 14 days.

TABLE 4
Post-lyophilization viability analysis data

| | Post- | Pre- | | |
|---|---|---|---|---|
| Combi- | lyophilization | lyophilization | % Decline | % |
| nations | % Viability | % Viability | in Viability | Viability |
| a | 46.09 | 78.96 | 32.87 | 58.37 |
| b | 28.83 | | 50.13 | 36.51 |
| c | 45.94 | | 33.02 | 58.18 |
| d** | 51.12 | | 27.84 | 64.74 |
| e | 55.29 | | 23.67 | 70.00 |
| f | 60 | | 18.96 | 75.98 |
| g | 51.27 | | 27.69 | 64.93 |
| h | 39.01 | | 39.95 | 49.40 |
| i | 49.42 | | 29.54 | 62.58 |
| j | 38.67 | | 40.29 | 48.97 |

% cell viability is calculated considering pre-lyophilisation viability as 100%; i.e., % cell viability = (post lyophylization % viability × 100)/(pre-lyophilization % viability)
% decline viability is calculated by subtracting post-lyophylization % viability from pre-lyophylization % viability.
**Combination ‘d’—showed very low cell count after 7AAD viability staining. Only ~2000 events captured.

### Example 2

Similar to Example 1, the effect of lyophilization on Mesenchymal Stem Cells (MSCs) was studied by exposing cells to different lyophilization protocols in presence of various combinations of ingredients. The viability of cells was analyzed before and after lyophilization.

TABLE 5
Sample Details

| | Mesenchymal |
|---|---|
| Cells used | Stem Cells |
| Passage No. | P2 |

a. Cell Suspension Preparation

Cells were grown in growth medium (DMEM-LG) to attain 90% confluency. Upon attaining 90% confluence, the cells were exposed to 100 mM Trehalose in DMEM-LG for 24 hours at 37° C. Cells were then trypsinized and re-suspended in 9 different combinations of Lyophilization Solutions. One part of the cell suspension was used to perform pre-lyophilization viability and cell surface marker analysis by flow cytometry.

TABLE 6
Mesenchymal Stem Cells lyophilization
mixture in a combination of ingredients

| Sr. | combination of ingredients |
|---|---|
| a | 500 mM Trehalose |
| b | 5% HSA |
| c | 0.5% Glycerol |
| d | 2% Dextran |
| e | 1% PEG400 |
| f | 500 mM Trehalose + 5% HSA |
| g | 500 mM Trehalose + 5% HSA + |
| | 0.5% Glycerol |
| h | 500 mM Trehalose + 5% HSA + |
| | 0.5% Glycerol + 2% Dextran |
| i | 500 mM Trehalose + 5% HSA + |
| | 0.5% Glycerol + 2% Dextran + |
| | 1% PEG400 |
| j | Vehicle Control |

All solutions prepared in DMEM-LG w/o Phenol red, Glucose and L-Glutamine (Vehicle).

TABLE 7
Pre-lyophilization viability and cell
surface marker analysis by flow cytometry

| | |
|---|---|
| Viability by 7AAD | 66.71% |
| CD90 | 80.12% |
| CD73 | 91.20% |
| CD105 | 38.75% |

As can be seen from above table, the observed pre-lyophilization cell viability analysed by 7AAD staining is 66.71%. The cell surface marker analysis shows that 80.12% of the cell population expresses CD90 expression, 91.20% of the cell population express CD73 expression and 38.75% of the cell population expresses CD105 expression.

Each combination of mesenchymal stem cells lyophilization mixtures as per table 6 was lyophilized. After lyophilization, the lyophilized products were sealed with 20 mm aluminium flip of seals and stored at 2-8° C. for a period of minimum 14 days.

b. Post-Lyophylization Viability Analysis by Flow Cytometry:

Viability of the lyophilized cells was analyzed by 7AAD staining 17 days post lyophilization. Briefly, each combination of the lyophilized product was individually reconstituted in 1×PBS and cells were centrifuged at 300 g to obtain a pellet. The pellet was re-suspended in 1×PBS. The cells were then stained with 7AAD dye to analyze cell viability by flow cytometry.

TABLE 8
Post-lyophilization viability analysis data

| | Post- | Pre- | | |
|---|---|---|---|---|
| Combi- | lyophilization | lyophilization | % Decline | % |
| nations | % Viability | % Viability | in Viability | Viability |
| a | 15.90 | 66.71 | 50.10 | 23.83 |
| b | 15.30 | | 50.70 | 22.93 |
| c | 24.10 | | 41.90 | 36.12 |
| d | 19.20 | | 46.80 | 28.78 |
| e | 39.70 | | 26.30 | 59.51 |
| f | 20.30 | | 45.70 | 30.43 |
| g | 18.40 | | 47.60 | 27.58 |
| h | 23.60 | | 42.40 | 35.37 |
| i | 22.10 | | 43.90 | 33.12 |
| j | 21.90 | | 44.10 | 32.82 |

% cell viability is calculated considering pre-lyophilisation viability as 100%; i.e., % cell viability = (post lyophylization % viability × 100)/(pre-lyophilization % viability)
% decline viability is calculated by subtracting post-lyophylization % viability from pre-lyophylization % viability.

Noting the initial results obtained in Example 1 and Example 2 various experiments were planned and performed, the following examples provide further detail in connection with what are presently deemed to be the most practical and preferred embodiments of the invention.

### Example 3

Similar to Example 1 and Example 2, the effect of lyophilization on Mesenchymal Stem Cells (MSCs) was studied by exposing cells to different lyophilization protocols in presence of various combinations of ingredients. The viability of cells was analyzed before and after lyophilization.

TABLE 9
Sample Details

| | Mesenchymal |
|---|---|
| Cells used | Stem Cells |
| Passage No. | P5 |

Cells were grown in growth medium (DMEM-LG) to attain 90% confluency. Upon attaining 90% confluence, the cells were exposed to 100 mM Trehalose in DMEM-LG for 24 hours at 37° C. Cells were then trypsinized and re-suspended in 6 combinations of Lyophilization Solutions. One part of the cell suspension was used to perform pre-lyophilization viability and cell surface marker analysis by flow cytometry.

TABLE 10
Combinations of lyophilization solutions

| Combi- | Components | | |
|---|---|---|---|
| nation # | Trehalose | H.S.A. | Dextran |
| 3A | 500 mM | — | — |
| 3B | — | 4% | — |
| 3C | — | 5% | — |
| 3D | 150 mM | 5% | — |
| 3E | 150 mM | — | 8% |
| 3F | 500 mM | — | 8% |

| | |
|---|---|
| 3G | Control (1X PBS) |

All solutions prepared in DMEM-LG w/o Phenol red, Glucose and L-Glutamine (Vehicle).

### Pre-Lyophilization Viability and Cell Surface Marker Analysis by Flow Cytometry

TABLE 11
Pre-lyophilization viability
by flow cytometry

| | |
|---|---|
| Viability by 7AAD | 100% |

As can be seen from above table, the observed pre-lyophilization cell viability analysed by 7AAD staining is 100%.

Each combination of mesenchymal stem cells lyophilization mixtures as described in table 10 was lyophilized. After lyophilization, vials containing the lyophilized products were sealed and stored at 2-8° C. for a period of minimum 14 days.

### Post-Lyophilization Viability and Cell-Surface Marker Analysis by Flow Cytometry:

Viability of one set of lyophilized cells was analysed by 7AAD staining 2 days post lyophilization. MSC-specific surface markers were analysed by staining the cells with corresponding antibodies.

Briefly, each combination of the lyophilized product was individually reconstituted in 1×PBS and cells were centrifuged at 300 g to obtain a pellet. The pellet was re-suspended in 1×PBS. One part of cells was then stained with 7AAD dye to analyse cell viability by flow cytometry (results of which are shown in Table 12). Remaining cells were stained with MSC specific surface marker antibodies (results of which are shown in Table 13).

TABLE 12
Post-lyophilization viability analysis data

| | Post- | Pre- | |
|---|---|---|---|
| Combi- | lyophilization | lyophilization | % Decline |
| nation # | % Viability | % Viability | in Viability |
| 3A | 83.40% | 100% | 16.60% |
| 3B | 85.20% | | 14.80% |
| 3C | 85.80% | | 14.20% |
| 3D | 86.40% | | 13.60% |
| 3E | 81.50% | | 18.50% |
| 3F | 69.70% | | 30.30% |
| 3G | 24.70% | | 75.30% |

TABLE 13
MSC surface marker analysis data

| | % Cells expressing | | |
|---|---|---|---|
| Combi- | the marker | | |
| nation # | CD45 | CD90 | CD73 |
| 3A | 0% | 98.6% | 96.5% |
| 3B | 0.1% | 91.7% | 81.7% |
| 3C | −0.4% | 98.4% | 93.3% |
| 3D | 0% | 91.9% | 57.60% |
| 3E | −2.3% | 95.8% | 92.5% |
| 3F | −1.1% | 93.4% | 93.40% |
| 3G | −1.5% | 99.6% | 98.2% |

### Example 4

Pre-lyophilization viability and cell surface marker analysis for below 7 combinations of Lyophilization Solutions by flow cytometry was performed (at Passage No: P4) according to the procedure described in Example 3.

TABLE 14
Combinations of lyophilization solutions

| Combi- | Components | | |
|---|---|---|---|
| nation # | Trehalose | HSA | Dextran |
| 4A | 500 mM | — | — |
| 4B | — | 4% | — |
| 4C | 150 mM | 5% | — |
| 4D | 500 mM | 10% | — |
| 4E | — | — | 1% |
| 4F | 150 mM | — | 8% |
| 4G | 300 mM | — | 8% |

| | |
|---|---|
| 4H | Control (1X PBS) |

All solutions prepared in DMEM-LG w/o Phenol red, Glucose and L-Glutamine (Vehicle).

TABLE 15
Pre-lyophilization viability and cell
surface marker analysis by flow cytometry

| | |
|---|---|
| Viability by 7AAD | 100% |
| Cell surface marker—CD45 | Not Done |
| Cell surface marker—CD90 | 99.3% |
| Cell surface marker—CD73 | 99.1% |

### Post-Lyophilization Viability and Cell-Surface Marker Analysis by Flow Cytometry

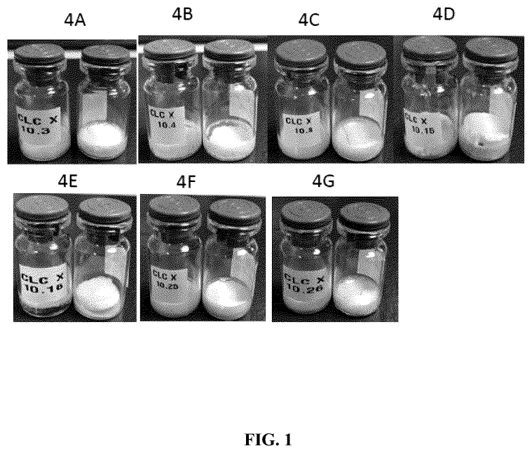

10 days post lyophilisation Viability and MSC-specific surface markers were analysed according to the procedure described in Example 3. FIG. 1 shows representative images of lyophilized cakes of combinations 4A to 4G.

TABLE 16
Post-lyophilization viability analysis data

| | Post- | Pre- | |
|---|---|---|---|
| Combi- | lyophilization | lyophilization | % Decline |
| nation # | % Viability | % Viability | in Viability |
| 4A | 73.50% | 100% | 26.50% |
| 4B | 87.90% | | 12.10% |
| 4C | 72% | | 28.00% |
| 4D | 45.10% | | 54.90% |
| 4E | 86.80% | | 13.20% |
| 4F | 91.70% | | 8.30% |
| 4G | 83.60% | | 16.40% |
| 4H | 14.30% | | 85.70% |

TABLE 17
MSC surface marker analysis data

| | % Cells expressing | | |
|---|---|---|---|
| Combi- | the marker | | |
| nation # | CD45 | CD90 | CD73 |
| 4A | −1.6% | 94.6% | 81.7% |
| 4B | 1.1% | 95.8% | 87.7% |
| 4C | 0.1% | 93.5% | 83.3% |
| 4D | 0.2% | 95.1% | 91.2% |
| 4E | 0% | 92.4% | 92.2% |
| 4F | −0.2% | 95.8% | 90.6% |
| 4G | 0.2% | 95.8% | 88.8% |
| 4H | −0.1% | 98.9% | 78.1% |

### Example 5

Pre-lyophilization viability and cell surface marker analysis for below 24 combinations of Lyophilization Solutions by flow cytometry was performed (at Passage No: P8) according to the procedure described in Example 3.

TABLE 18
Combinations of lyophilization solutions

| Combi- | Components | | | |
|---|---|---|---|---|
| nation # | Trehalose | H.S.A. | Dextran | PEG400 |
| 5A | 500 mM | 4% | — | — |
| 5B | — | 4% | 1% | — |
| 5C | — | 4% | — | 0.1% |
| 5D | 500 mM | 4% | 1% | — |
| 5E | — | 4% | 1% | 0.1% |
| 5F | 500 mM | 4% | — | 0.1% |
| 5G | 500 mM | 4% | 1% | 0.1% |
| 5H | — | 5% | 1% | — |
| 5I | — | 5% | — | 0.1% |
| 5J | — | 5% | 1% | 0.1% |
| 5K | — | — | 8% | — |
| 5L | — | 5% | 8% | — |
| 5M | — | — | 8% | 0.1% |
| 5N | — | 5% | 8% | 0.1% |
| 50 | 500 mM | — | 8% | 0.1% |
| 5P | 500 mM | 5% | 8% | 0.1% |
| 5Q | — | — | 10% | — |
| 5R | 500 mM | — | 10% | — |
| 5S | — | 5% | 10% | — |
| 5T | — | — | 10% | 0.1% |
| 5U | 500 mM | 5% | 10% | |
| 5V | — | 5% | 10% | 0.1% |
| 5W | 500 mM | — | 10% | 0.1% |
| 5X | 500 mM | 5% | 10% | 0.1% |

| | |
|---|---|
| 5Y | Vehicle Control |

All solutions prepared in DMEM-LG w/o Phenol red, Glucose and L-Glutamine (Vehicle).

### Pre-Lyophilization Viability and Cell Surface Marker Analysis by Flow Cytometry

TABLE 19
Pre-lyophilization viability and cell
surface marker analysis by flow cytometry

| | |
|---|---|
| Viability by 7AAD | 98.1% |
| Cell surface marker—CD45 | 0.1% |
| Cell surface marker—CD90 | 98.4% |
| Cell surface marker—CD73 | 99.0% |

### Post-Lyophilization Viability and Cell-Surface Marker Analysis by Flow Cytometry

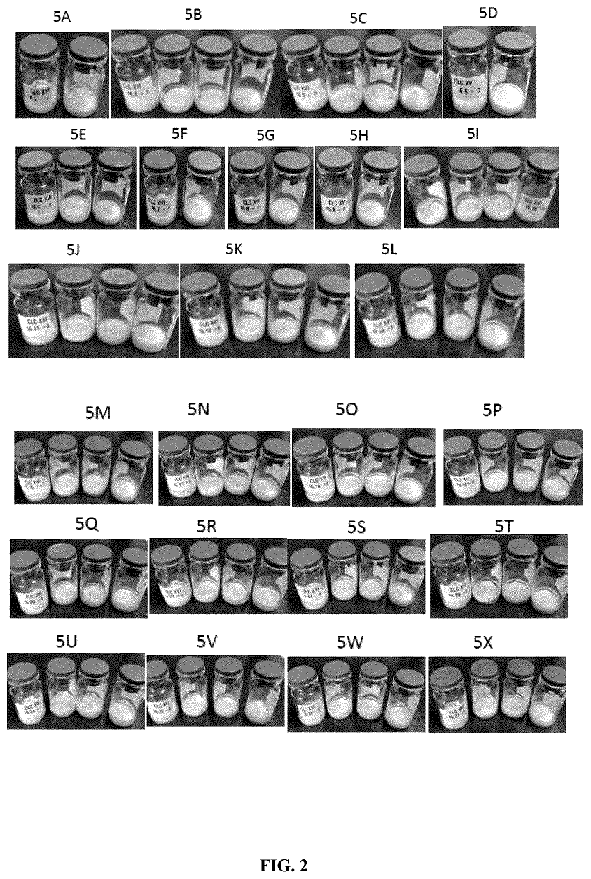

15 days post lyophilisation Viability and MSC-specific surface markers were analysed according to the procedure described in Example 3. FIG. 2 shows representative images of lyophilized cakes of combinations 5A to 5X.

TABLE 20
Post-lyophilization viability analysis data

| | Post- | Pre- | |
|---|---|---|---|
| Combi- | lyophilization | lyophilization | % Decline |
| nation # | % Viability | % Viability | in Viability |
| 5A | 49.50% | 98.10% | 48.60% |
| 5B | 81.10% | | 17.00% |
| 5C | 86.50% | | 11.60% |
| 5D | 59.20% | | 38.90% |
| 5E | 84.90% | | 13.20% |
| 5F | 45.50% | | 52.60% |
| 5G | 43.90% | | 54.20% |
| 5H | 80.90% | | 17.20% |
| 5I | 94.60% | | 3.50% |
| 5J | 71.40% | | 26.70% |
| 5K | 36.70% | | 61.40% |
| 5L | 30% | | 68.10% |
| 5M | 36.20% | | 61.90% |
| 5N | 27.60% | | 70.50% |
| 50 | 49.20% | | 48.90% |
| 5P | 42.30% | | 55.80% |
| 5Q | 93.30% | | 4.80% |
| 5R | 91.90% | | 6.20% |
| 5S | 39.70% | | 58.40% |
| 5T | 36.80% | | 61.30% |
| 5U | 50.20% | | 47.90% |
| 5V | 40% | | 58.10% |
| 5W | 43.40% | | 54.70% |
| 5X | 46% | | 52.10% |
| 5Y | 13.50% | | 84.60% |

TABLE 21
MSC surface marker analysis data

| | % Cells expressing the marker | | |
|---|---|---|---|
| Combination # | CD45 | CD90 | CD73 |
| 5A | −0.9% | 98.7% | 95.70% |
| 5B | −0.5% | 96.8% | 81.80% |
| 5C | −0.5% | 97.8% | 83.20% |
| 5D | 0.2% | 94% | 89.70% |
| 5E | 0.1% | 89.8% | 78.40% |
| 5F | −0.3% | 92% | 86.20% |
| 5G | 0% | 92.3% | 88.40% |
| 5H | 0.6% | 89.6% | 80.10% |
| 5I | 0.6% | 91.7% | 88.40% |
| 5J | 0.7% | 90.3% | 81.30% |
| 5K | −0.4% | 90.6% | 84.00% |
| 5L | −1% | 98% | 94.40% |
| 5M | 0.5% | 92.6% | 86.50% |
| 5N | 0.1% | 98.7% | 95.40% |
| 5O | 0.1% | 97.7% | 97.60% |
| 5P | 0% | 98.8% | 97.50% |
| 5Q | 0.2% | 87.5% | 94.00% |
| 5R | 0.2% | 95.1% | 91.2% |
| 5S | −0.1% | 95.4% | 91.90% |
| 5T | 0.2% | 93.8% | 89.80% |
| 5U | 0% | 96.6% | 93.70% |
| 5V | 0.3% | 96.5% | 94.20% |
| 5W | 0.2% | 96.3% | 92.80% |
| 5X | 0.1% | 97.1% | 94.90% |
| 5Y | −0.1% | 99.2% | 98.30% |

### Example 6

Pre-lyophilization viability and cell surface marker analysis for below 25 combinations of Lyophilization Solutions by flow cytometry was performed (at Passage No: P6) according to the procedure described in Example 3.

TABLE 22
Combinations of lyophilization solutions

| | Components | | | |
|---|---|---|---|---|
| Combination # | Trehalose | H.S.A. | Dextran | PEG400 |
| 6A | 500 mM | — | — | — |
| 6B | — | 5% | — | — |
| 6C | 150 mM | 5% | — | — |
| 6D | — | 4% | 4% | — |
| 6E | — | 5% | 4% | — |
| 6F | — | 10% | 4% | — |
| 6G | — | 4% | 8% | — |
| 6H | — | 5% | 8% | — |
| 6I | — | 10% | 8% | — |
| 6J | 150 mM | 4% | 4% | — |
| 6K | 150 mM | 10% | 4% | — |
| 6L | 150 mM | 4% | 8% | — |
| 6M | 150 mM | 5% | 8% | — |
| 6N | 150 mM | 10% | 8% | — |
| 6O | 500 mM | 4% | 4% | — |
| 6P | 500 mM | 10% | 4% | — |
| 6Q | 500 mM | 4% | 8% | — |
| 6R | 500 mM | 5% | 8% | — |
| 6S | 500 mM | 10% | 8% | — |
| 6T | — | — | — | 2% |
| 6U | — | — | — | 4% |
| 6V | — | — | — | 8% |
| 6W | — | — | 8% | 2% |
| 6X | — | — | 8% | 4% |
| 6Y | — | — | 8% | 8% |

| | |
|---|---|
| 6Z | Control (1X PBS) |

All solutions prepared in DMEM-LG w/o Phenol red, Glucose and L-Glutamine (Vehicle).

### Pre-Lyophilization Viability and Cell Surface Marker Analysis by Flow Cytometry

TABLE 23
Pre-lyophilization viability and cell surface marker analysis by
flow cytometry

| | |
|---|---|
| Viability by 7AAD | 97.7% |
| Cell surface marker - CD45 | −0.1% |
| Cell surface marker - CD90 | 94.7% |
| Cell surface marker - CD73 | 97.4% |

### Post-Lyophilization Viability and Cell-Surface Marker Analysis by Flow Cytometry

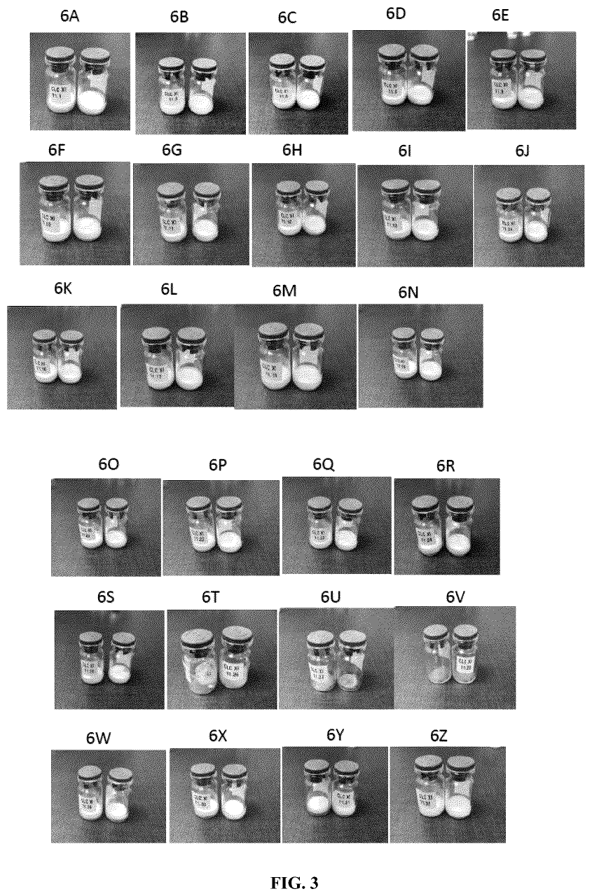

16 days post lyophilisation Viability (Table 24) and MSC-specific surface markers (Table 25) were analysed according to the procedure described in Example 3. FIG. 3 shows representative images of lyophilized cakes of combinations 6A to 6Z.

TABLE 24
Post-lyophilization viability analysis data

| | Post-lyophilization | Pre-lyophilization | % Decline in |
|---|---|---|---|
| Combination # | % Viability | % Viability | Viability |
| 6A | 53.30% | 97.70% | 44.40% |
| 6B | 83.90% | | 13.80% |
| 6C | 70.80% | | 26.90% |
| 6D | 50.50% | | 47.20% |
| 6E | 53.70% | | 44.00% |
| 6F | 48.50% | | 49.20% |
| 6G | 50.20% | | 47.50% |
| 6H | 58.40% | | 39.30% |
| 6I | 39.40% | | 58.30% |
| 6J | 52.90% | | 44.80% |
| 6K | 43.80% | | 53.90% |
| 6L | 44.20% | | 53.50% |
| 6M | 37.80% | | 59.90% |
| 6N | 42.70% | | 55.00% |
| 6O | 57% | | 40.70% |
| 6P | 49% | | 48.70% |
| 6Q | 55.50% | | 42.20% |
| 6R | 48.80% | | 48.90% |
| 6S | 40.80% | | 56.90% |
| 6T | 28.90% | | 68.80% |
| 6U | 26.40% | | 71.30% |
| 6V | 32.20% | | 65.50% |
| 6W | 38.90% | | 58.80% |
| 6X | 35.70% | | 62.00% |
| 6Y | 30% | | 67.70% |
| 6Z | 23.60% | | 74.10% |

TABLE 25
MSC surface marker analysis data

| | % Cells expressing the marker | | |
|---|---|---|---|
| Combination # | CD45 | CD90 | CD73 |
| 6A | 0.1% | 91.7% | 75.60% |
| 6B | −0.1% | 93.5% | 88.80% |
| 6C | −0.5% | 98.6% | 92.2% |
| 6D | −0.5% | 96.8% | 81.80% |
| 6E | 0.6% | 89.6% | 80.10% |
| 6F | 0.1% | 37.7% | 44.40% |
| 6G | −0.1% | 89.4% | 86.30% |
| 6H | −1% | 98% | 94.40% |
| 6I | −0.1% | 93.3% | 90.40% |
| 6J | −0.2% | 90.8% | 85.00% |
| 6K | 0% | 92.4% | 46.30% |
| 6L | 0.4% | 97.4% | 93.70% |
| 6M | −0.1% | 92.2% | 91.00% |
| 6N | −0.2% | 90.8% | 85.00% |
| 6O | −0.4% | 94.4% | 91.40% |
| 6P | 0% | 92.2% | 89.20% |
| 6Q | −1.1% | 93.4% | 93.40% |
| 6R | 0.2% | 99.3% | 98.50% |
| 6S | 0.3% | 97.5% | 93.70% |
| 6T | −2.4% | 95.8% | 93.20% |
| 6U | 0.1% | 97.8% | 95.80% |
| 6V | −0.4% | 90.6% | 84.00% |
| 6W | 0.5% | 92.6% | 86.50% |
| 6X | −1.6% | 97.7% | 94.80% |
| 6Y | −1% | 98% | 94.40% |
| 6Z | −1.5% | 94.1% | 63.5% |

### Example 7

Pre-lyophilization viability and cell surface marker analysis for below 23 combinations of Lyophilization Solutions by flow cytometry was performed (at Passage No: P6) according to the procedure described in Example 3.

TABLE 26
Combinations of lyophilization solutions

| | Components | | | |
|---|---|---|---|---|
| Combination # | Trehalose | H.S.A. | Dextran | PEG400 |
| 7A | — | — | — | 0.1% |
| 7B | 500 mM | 5% | — | — |
| 7C | 500 mM | — | 1% | — |
| 7D | 500 mM | — | — | 0.1% |
| 7E | 500 mM | 5% | 1% | — |
| 7F | 500 mM | — | 1% | 0.1% |
| 7G | 500 mM | 5% | — | 0.1% |
| 7H | 500 mM | 5% | 1% | 0.1% |
| 7I | 300 mM | — | — | — |
| 7J | 300 mM | 5% | — | — |
| 7K | 300 mM | — | 1% | — |
| 7L | 300 mM | — | — | 0.1% |
| 7M | 300 mM | 5% | 1% | — |
| 7N | 300 mM | — | 1% | 0.1% |
| 7O | 300 mM | 5% | — | 0.1% |
| 7P | 300 mM | 5% | 1% | 0.1% |
| 7Q | 150 mM | — | — | — |
| 7R | 150 mM | — | 1% | — |
| 7S | 150 mM | — | — | 0.1% |
| 7T | 150 mM | 5% | 1% | — |
| 7U | 150 mM | — | 1% | 0.1% |
| 7V | 150 mM | 5% | — | 0.1% |
| 7W | 150 mM | 5% | 1% | 0.1% |

| | |
|---|---|
| 7X | Control - 1X PBS |

All solutions prepared in DMEM-LG w/o Phenol red, Glucose and L-Glutamine (Vehicle).

### Pre-Lyophilization Viability and Cell Surface Marker Analysis by Flow Cytometry

TABLE 27
Pre-lyophilization viability and cell surface marker analysis by
flow cytometry

| | |
|---|---|
| Viability by 7AAD | 98.1% |
| Cell surface marker - CD45 | 0.7% |
| Cell surface marker - CD90 | 97.7% |
| Cell surface marker - CD73 | 98.8% |

### Post-Lyophilization Viability and Cell-Surface Marker Analysis by Flow Cytometry

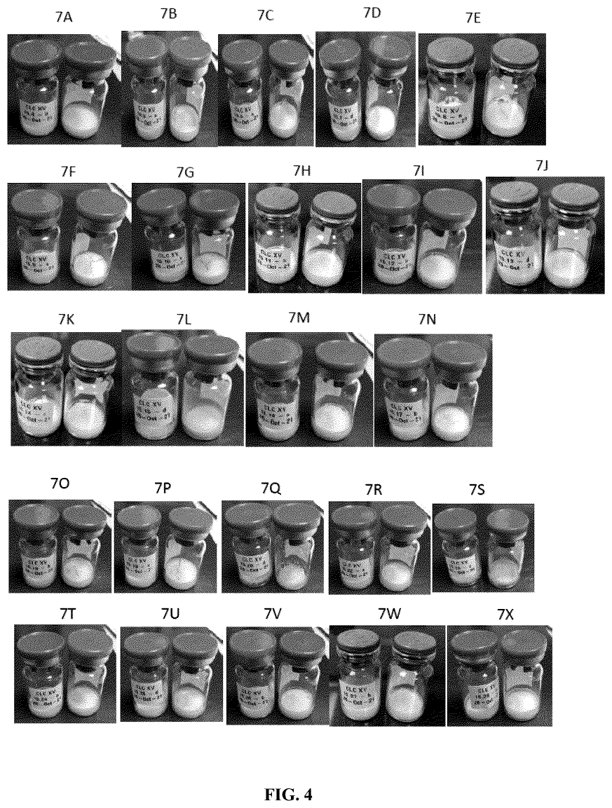

18 days post lyophilisation Viability and MSC-specific surface markers were analysed according to the procedure described in Example 3. FIG. 4 shows representative images of lyophilized cakes of combinations 7A to 7X.

TABLE 28
Post-lyophilization viability analysis data

| | Post-lyophilization | Pre-lyophilization | % Decline in |
|---|---|---|---|
| Combination # | % Viability | % Viability | Viability |
| 7A | 79.60% | 98.10% | 18.50% |
| 7B | 92.10% | | 6.00% |
| 7C | 95.70% | | 2.40% |
| 7D | 95.20% | | 2.90% |
| 7E | 89.60% | | 8.50% |
| 7F | 93.10% | | 5.00% |
| 7G | 92.90% | | 5.20% |
| 7H | 89.60% | | 8.50% |
| 7I | 91.70% | | 6.40% |
| 7J | 93.30% | | 4.80% |
| 7K | 94.50% | | 3.60% |
| 7L | 97.20% | | 0.90% |
| 7M | 95.80% | | 2.30% |
| 7N | 97.10% | | 1.00% |
| 7O | 96% | | 2.10% |
| 7P | 95.60% | | 2.50% |
| 7Q | 95.40% | | 2.70% |
| 7R | 94% | | 4.10% |
| 7S | 97.40% | | 0.70% |
| 7T | 93.40% | | 4.70% |
| 7U | 96% | | 2.10% |
| 7V | 93.80% | | 4.30% |
| 7W | 94.70% | | 3.40% |
| 7X | 37.60% | | 60.50% |

TABLE 29
MSC surface marker analysis data

| | % Cells expressing the marker | | |
|---|---|---|---|
| Combination # | CD45 | CD90 | CD73 |
| 7A | −2.4% | 95.8% | 93.20% |
| 7B | 0.5% | 97.3% | 95.80% |
| 7C | 0.4% | 97.3% | 96.90% |
| 7D | 0.5% | 97.5% | 97.30% |
| 7E | 0.3% | 98% | 96.20% |
| 7F | 1.2% | 96.6% | 95.20% |
| 7G | 0.2% | 97.8% | 97.00% |
| 7H | 0.2% | 97.6% | 97.00% |
| 7I | 1.6% | 93.8% | 91.40% |
| 7J | 0.5% | 96.8% | 93.70% |
| 7K | 1.3% | 95.7% | 93.10% |
| 7L | −0.3% | 96.6% | 94.50% |
| 7M | 0.3% | 97.5% | 93.70% |
| 7N | 0.9% | 95.7% | 91.70% |
| 7O | 1.6% | 98.1% | 95.60% |
| 7P | 0.3% | 97.4% | 94.50% |
| 7Q | 1.6% | 97.3% | 94.80% |
| 7R | 0% | 97.7% | 92.60% |
| 7S | 0.1% | 97.8% | 95.80% |
| 7T | 0.4% | 97.4% | 93.70% |
| 7U | 0.6% | 96.9% | 93.60% |
| 7V | −0.1% | 95.8% | 92.20% |
| 7W | 0.1% | 95.7% | 93.20% |
| 7X | 0.2% | 97.2% | 97.30% |

### Example 8

Pre-lyophilization viability and cell surface marker analysis for below 15 combinations of Lyophilization Solutions by flow cytometry was performed (at Passage No: P2) according to the procedure described in Example 3.

TABLE 30
Combinations of lyophilization solutions

| Combi- | Components | | | | | | |
|---|---|---|---|---|---|---|---|
| nation # | Trehalose | HSA | Glycerol | Dextran | PEG400 | PEG6K | DMSO |
| 8A | — | — | 0.20% | — | — | — | — |
| 8B | — | — | — | 4% | — | — | — |
| 8C | — | — | — | — | 0.50% | — | — |
| 8D | 500 mM | 5% | — | — | — | — | — |
| 8E | 500 mM | 5% | 0.20% | — | — | — | — |
| 8F | 500 mM | 5% | 0.20% | 4% | — | — | — |
| 8G | 500 mM | 5% | 0.20% | 4% | 0.50% | — | — |
| 8H | 250 mM | — | — | — | — | — | — |
| 8I | — | — | 0.30% | — | — | — | — |
| 8J | — | — | — | 1% | — | — | — |
| 8K | 250 mM | 5% | 0.30% | 1% | 0.50% | — | — |
| 8L | 250 mM | — | — | — | — | 0.50% | |
| 8M | Vehicle Control | | | | | | |
| 8N | 500 mM | 5% | 0.20% | 4% | 0.50% | — | 2.50% |
| 8O | 500 mM | 5% | 0.20% | 4% | 0.50% | — | 5.00% |
| 8P | 500 mM | 5% | 0.20% | 4% | 0.50% | — | 7.50% |

All solutions prepared in DMEM-LG w/o Phenol red, Glucose and L-Glutamine (Vehicle).

TABLE 31
Pre-lyophilization viability and cell surface marker analysis by
flow cytometry

| | |
|---|---|
| Viability by 7AAD | 95.15% |
| Cell surface marker - CD90 | 96.20% |
| Cell surface marker - CD73 | 98.75% |

### Post-Lyophilization Viability and Cell-Surface Marker Analysis by Flow Cytometry

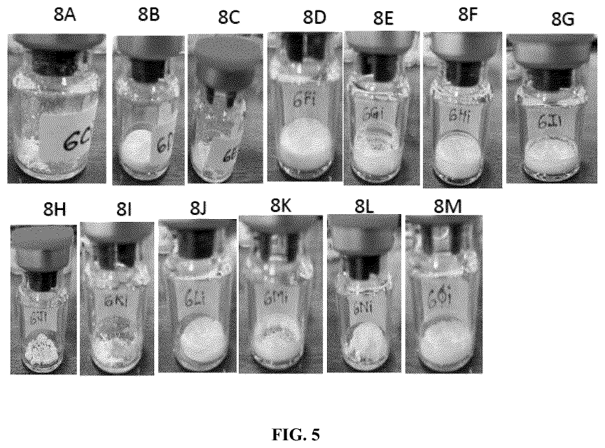

24 days post lyophilisation Viability and MSC-specific surface markers were analysed according to the procedure described in Example 3. For combinations 8N, 8O and 8P, the lyophilized product showed blow-out. FIG. 5 shows representative images of lyophilized cakes of combinations 8A to 8M.

TABLE 32
Post-lyophilization viability analysis data

| | Post-lyophilization | Pre-lyophilization | % Decline in |
|---|---|---|---|
| Combination # | % Viability | % Viability | Viability |
| 8A | 41.40% | 95.15% | 53.75% |
| 8B | 57% | | 38.15% |
| 8C | 94.70% | | 0.45% |
| 8D | 56.50% | | 38.65% |
| 8E | 64.50% | | 30.65% |
| 8F | 43.30% | | 51.85% |
| 8G | 59% | | 36.15% |
| 8H | 69.90% | | 25.25% |
| 8I | 51.40% | | 43.75% |
| 8J | 65.30% | | 29.85% |
| 8K | 74.60% | | 20.55% |
| 8L | 81.40% | | 13.75% |
| 8M | 76.60% | | 18.55% |
| 8N | N.D. | | — |
| 8O | N.D. | | — |
| 8P | N.D. | | — |

TABLE 33
MSC surface marker analysis data

| | % Cells expressing the marker | |
|---|---|---|
| Combination # | CD90 | CD73 |
| 8A | 97.4% | 95.5% |
| 8B | 90.6% | 84.00% |
| 8C | 97.8% | 95.80% |
| 8D | 88.4% | 60.00% |
| 8E | 97.8% | 97.00% |
| 8F | 99.3% | 98.50% |
| 8G | 98.8% | 97.50% |
| 8H | 90.8% | 85.00% |
| 8I | −2.4% | 95.8% |
| 8J | 93.5% | 90.60% |
| 8K | 92.3% | 91.00% |
| 8L | 95.7% | 93.10% |
| 8M | 99.2% | 98.30% |

| | |
|---|---|
| 8N | Not tested* |
| 8O | Not tested* |
| 8P | Not tested* |

*For combinations 8N, 8O, and 8P, the lyophilized product showed blow-out. Hence, post-lyophilization flow was not done for those combinations.

### Example 9

Pre-lyophilization viability and cell surface marker analysis for below 10 combinations of Lyophilization Solutions by flow cytometry was performed (at Passage No: P5) according to the procedure described in Example 3.

TABLE 34
Combinations of lyophilization solutions

| Combi- | Components | | | | | |
|---|---|---|---|---|---|---|
| nation # | Trehalose | HSA | Glycerol | Dextran | PEG400 | PEG600 |
| 9A | — | — | 0.20% | — | — | — |
| 9B | — | — | — | 4% | — | — |
| 9C | — | — | — | — | 0.50% | — |
| 9D | 500 mM | 5% | — | — | — | — |
| 9E | 500 mM | 5% | 0.20% | — | — | — |
| 9F | 500 mM | 5% | 0.20% | 4% | — | — |
| 9G | 500 mM | 5% | 0.20% | 4% | 0.50% | — |
| 9H | 250 mM | — | — | — | — | — |
| 9I | 250 mM | 5% | 0.30% | 1% | 0.50% | — |
| 9J | 250 mM | — | — | — | — | 0.50% |

| | |
|---|---|
| 9K | Vehicle Control |

All solutions prepared in DMEM-LG w/o Phenol red, Glucose and L-Glutamine (Vehicle).

TABLE 35
Pre-lyophilization viability and cell surface marker analysis by
flow cytometry

| | |
|---|---|
| Viability by 7AAD | 98.90% |
| Cell surface marker - CD90 | 99.40% |
| Cell surface marker - CD73 | 99.52% |

### Post-Lyophilization Viability and Cell-Surface Marker Analysis by Flow Cytometry

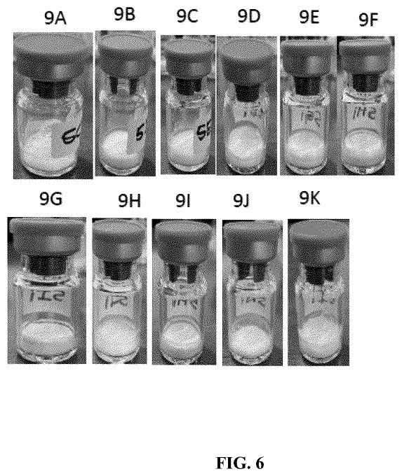

30 days post lyophilisation Viability and MSC-specific surface markers were analysed according to the procedure described in Example 3. FIG. 6 shows representative images of lyophilized cakes of combinations 9A to 9K.

TABLE 36
Post-lyophilization viability analysis data

| | Post-lyophilization | Pre-lyophilization | % Decline in |
|---|---|---|---|
| Combination | % Viability | % Viability | Viability |
| 9A | 66.20% | 98.90% | 32.70% |
| 9B | 72.10% | | 26.80% |
| 9C | 83% | | 15.90% |
| 9D | 43.10% | | 55.80% |
| 9E | 52.90% | | 46.00% |
| 9F | 68.70% | | 30.20% |
| 9G | 73.30% | | 25.60% |
| 9H | 45.20% | | 53.70% |
| 9I | 48.10% | | 50.80% |
| 9J | 48% | | 50.90% |
| 9K | 66% | | 32.90% |

TABLE 37
MSC surface marker analysis data

| | % Cells expressing the marker | |
|---|---|---|
| Combination # | CD90 | CD73 |
| 9A | 96.6% | 88.10% |
| 9B | 97.9% | 92.20% |
| 9C | 95.8% | 93.20% |
| 9D | 97.3% | 95.80% |
| 9E | 98% | 96.20% |
| 9F | 97.6% | 97.00% |
| 9G | 92.3% | 88.40% |
| 9H | 93.8% | 91.40% |
| 9I | 97.4% | 94.50% |
| 9J | 96.6% | 94.50% |
| 9K | 97.2% | 97.30% |

### Example 10

Pre-lyophilization viability and cell surface marker analysis for below 2 combinations of Lyophilization Solutions by flow cytometry was performed (at Passage No: P4) according to the procedure described in Example 3.

TABLE 38
Combinations of lyophilization solutions

| | Components | | |
|---|---|---|---|
| Combination # | Trehalose | HSA | Dextran |
| 10A | — | — | 1% |
| 10B | 150 mM | — | 8% |

| | |
|---|---|
| 10C | Control (1X PBS) |

All solutions prepared in DMEM-LG w/o Phenol red, Glucose and L-Glutamine (Vehicle).

TABLE 39
Pre-lyophilization viability and cell surface marker analysis by
flow cytometry

| | |
|---|---|
| Viability by 7AAD | 99% |
| Cell surface marker - CD45 | −0.2% |
| Cell surface marker - CD90 | 99.1% |
| Cell surface marker - CD73 | 99.4% |

### Post-Lyophilization Viability and Cell-Surface Marker Analysis by Flow Cytometry

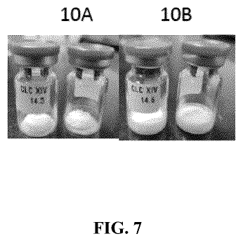

30 days post lyophilisation Viability and MSC-specific surface markers were analysed according to the procedure described in Example 3. FIG. 7 shows representative images of lyophilized cakes of combinations 10A and 10B.

TABLE 40
Post-lyophilization viability analysis data

| | Post-lyophilization | Pre-lyophilization | % Decline in |
|---|---|---|---|
| Combination # | % Viability | % Viability | Viability |
| 10A | 72.90% | 99.0% | 26.10% |
| 10B | 77.00% | | 22.00% |
| 10C | 29.43% | | 69.57% |

TABLE 41
MSC surface marker analysis data

| | % Cells expressing the marker | | |
|---|---|---|---|
| Combination # | CD45 | CD90 | CD73 |
| 10A | −0.2% | 97.4% | 95.5% |
| 10B | −1.1% | 98.9% | 92.6% |
| 10C | 2.6% | 99.6% | 98.2% |

### Example 11

Pre-lyophilization viability and cell surface marker analysis for below 23 combinations of Lyophilization Solutions by flow cytometry was performed (at Passage No: P3) according to the procedure described in Example 3.

TABLE 42
Combinations of lyophilization solutions

| Combi- | Components | | | | | |
|---|---|---|---|---|---|---|
| nation # | Trehalose | HSA | Glycerol | Dextran | PEG400 | PEG600 |
| 11A | 150 mM | — | — | — | — | — |
| 11B | — | — | 0.10% | — | — | — |
| 11C | — | — | — | 4% | — | — |
| 11D | — | — | — | — | 0.50% | — |
| 11E | — | — | — | — | — | 0.10% |
| 11F | 500 mM | 5% | — | — | — | — |
| 11G | 500 mM | 5% | 0.10% | — | — | — |
| 11H | 500 mM | 5% | 0.10% | 4% | — | — |
| 11I | 500 mM | 5% | 0.10% | 4% | 0.50% | — |
| 11J | 500 mM | 5% | 0.10% | 4% | 0.50% | 0.10% |
| 11K | 500 mM | 5% | — | 4% | — | — |
| 11L | 500 mM | 5% | — | — | 0.50% | — |
| 11M | 500 mM | 5% | — | — | — | — |
| 11N | 500 mM | 5% | — | 4% | 1% | — |
| 11O | 500 mM | 5% | — | 4% | — | — |
| 11P | 150 mM | 5% | — | 4% | — | — |
| 11Q | 150 mM | 5% | — | 4% | 1% | — |
| 11R | 150 mM | 5% | 0.10% | — | — | — |
| 11S | 150 mM | 5% | 0.10% | 4% | — | — |
| 11T | 150 mM | 5% | 0.10% | 4% | 0.50% | — |
| 11U | 150 mM | 5% | 0.10% | 4% | 0.50% | 0.10% |

| | |
|---|---|
| 11V | Vehicle Control |

All solutions prepared in DMEM-LG w/o Phenol red, Glucose and L-Glutamine (Vehicle).

TABLE 43
Pre-lyophilization viability and cell surface marker analysis by
flow cytometry

| | |
|---|---|
| Viability by 7AAD | 95.6% |
| Cell surface marker - CD90 | 98.7% |
| Cell surface marker - CD73 | 99% |

### Post-Lyophilization Viability and Cell-Surface Marker Analysis by Flow Cytometry

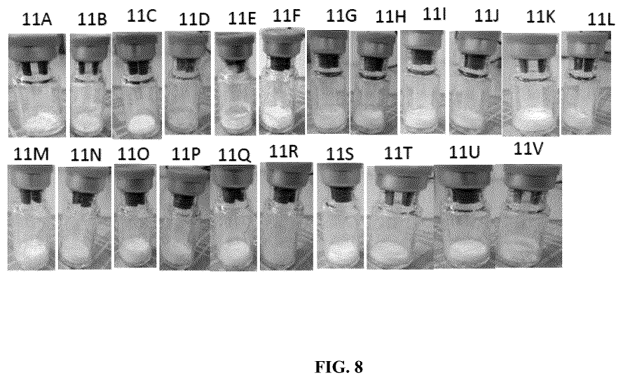

48 days post lyophilisation Viability and MSC-specific surface markers were analysed according to the procedure described in Example 3. FIG. 8 shows representative images of lyophilized cakes of combinations 11 A to 11V.

TABLE 44
Post-lyophilization viability analysis data

| | Post-lyophilization | Pre-lyophilization | % Decline in |
|---|---|---|---|
| Combination # | % Viability | % Viability | Viability |
| 11A | 71.60% | 95.6% | 24.00% |
| 11B | 59.70% | | 35.90% |
| 11C | 70.70% | | 24.90% |
| 11D | 86.20% | | 9.40% |
| 11E | 73.30% | | 22.30% |
| 11F | 61.20% | | 34.40% |
| 11G | 73.30% | | 22.30% |
| 11H | 79.90% | | 15.70% |
| 11I | 79.10% | | 16.50% |
| 11J | 77.40% | | 18.20% |
| 11K | 75% | | 20.60% |
| 11L | 72.80% | | 22.80% |
| 11M | 78.20% | | 17.40% |
| 11N | 66.30% | | 29.30% |
| 11O | 70% | | 25.60% |
| 11P | 70.20% | | 25.40% |
| 11Q | 76.50% | | 19.10% |
| 11R | 74.80% | | 20.80% |
| 11S | 67.70% | | 27.90% |
| 11T | 74.70% | | 20.90% |
| 11U | 67.50% | | 28.10% |
| 11V | 73.60% | | 22.00% |

TABLE 45
MSC surface marker analysis data

| | % Cells expressing the marker | |
|---|---|---|
| Combination # | CD90 | CD73 |
| 11A | 94% | 84.60% |
| 11B | 97.8% | 83.20% |
| 11C | 92.7% | 90.90% |
| 11D | 95.8% | 93.20% |
| 11E | 91.7% | 88.40% |
| 11F | 88.4% | 60.00% |
| 11G | 97.3% | 95.80% |
| 11H | 89.6% | 77.90% |
| 11I | 97.8% | 97.00% |
| 11J | 97.6% | 97.00% |
| 11K | 98% | 96.20% |
| 11L | 99.3% | 98.50% |
| 11M | 94.6% | 93.80% |
| 11N | 96.6% | 93.70% |
| 11O | 92.3% | 88.40% |
| 11P | 95.5% | 84.10% |
| 11Q | 97.7% | 92.60% |
| 11R | 97.4% | 93.70% |
| 11S | 96.9% | 93.60% |
| 11T | 95.8% | 92.20% |
| 11U | 95.7% | 93.20% |
| 11V | 81.5% | 82.10% |

### Example 12

Pre-lyophilization viability and cell surface marker analysis for below 6 combinations of Lyophilization Solutions by flow cytometry was performed (at Passage No: P3) according to the procedure described in Example 3.

TABLE 46
Combinations of lyophilization solutions

| | Components | | |
|---|---|---|---|
| Combination # | Trehalose | HSA | Dextran |
| 12A | 500 mM | — | — |
| 12B | — | 4% | — |
| 12C | — | 5% | — |
| 12D | 150 mM | 5% | — |
| 12E | — | — | 1% |
| 12F | 500 mM | — | 8% |

| | |
|---|---|
| 12G | Control (1X PBS) |

All solutions prepared in DMEM-LG w/o Phenol red, Glucose and L-Glutamine (Vehicle).

TABLE 47
Pre-lyophilization viability and cell surface marker analysis by
flow cytometry

| | |
|---|---|
| Viability by 7AAD | 98.8% |
| Cell surface marker - CD45 | −0.2% |
| Cell surface marker - CD90 | 98.6% |
| Cell surface marker - CD73 | 98.2% |

### Post-Lyophilization Viability and Cell-Surface Marker Analysis by Flow Cytometry

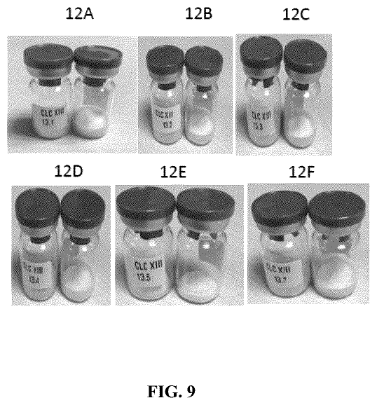

60 days post lyophilisation Viability and MSC-specific surface markers were analysed according to the procedure described in Example 3. FIG. 9 shows representative images of lyophilized cakes of combinations 12A to 12F.

TABLE 48
Post-lyophilization viability analysis data

| | Post-lyophilization | Pre-lyophilization | % Decline in |
|---|---|---|---|
| Combination # | % Viability | % Viability | Viability |
| 12A | 65.20% | 98.8% | 33.60% |
| 12B | 82.00% | | 16.80% |
| 12C | 80.90% | | 17.90% |
| 12D | 67.20% | | 31.60% |
| 12E | 68.90% | | 29.90% |
| 12F | 65.30% | | 33.50% |
| 12G | 29.50% | | 69.30% |

TABLE 49
MSC surface marker analysis data

| | % Cells expressing the marker | | |
|---|---|---|---|
| Combination # | CD45 | CD90 | CD73 |
| 12A | 0% | 95.8% | 92.6% |
| 12B | 0.1% | 91.7% | 81.7% |
| 12C | −0.4% | 92.4% | 83.3% |
| 12D | −0.5% | 94.6% | 87.7% |
| 12E | −1.6% | 98% | 93.8% |
| 12F | −0.4% | 92.4% | 87.0% |
| 12G | −1.5% | 94.1% | 63.5% |

### Example 13

Pre-lyophilization viability and cell surface marker analysis for below 4 combinations of Lyophilization Solutions by flow cytometry was performed at Passage No: P8 according to the procedure described in Example 3.

TABLE 50
Combinations of lyophilization solutions

| | Components | | |
|---|---|---|---|
| Combination # | Trehalose | HSA | Dextran |
| 13A | — | 5% | — |
| 13B | — | — | 1% |
| 13C | 150 mM | — | 8% |
| 13D | 500 mM | — | 8% |

| | |
|---|---|
| 13E | Control (1X PBS) |

All solutions prepared in DMEM-LG w/o Phenol red, Glucose and L-Glutamine (Vehicle).

TABLE 51
Pre-lyophilization viability and cell surface marker analysis by
flow cytometry

| | |
|---|---|
| Viability by 7AAD | 100% |
| Cell surface marker - CD45 | Not Done |
| Cell surface marker - CD90 | 99.3% |
| Cell surface marker - CD73 | 99.1% |

### Post-Lyophilization Viability and Cell-Surface Marker Analysis by Flow Cytometry

165 days (5.5 Months) post lyophilisation Viability and MSC-specific surface markers were analysed according to the procedure described in Example 3.

TABLE 52
Post-lyophilization viability analysis data

| | Post-lyophilization | Pre-lyophilization | % Decline in |
|---|---|---|---|
| Combination # | % Viability | % Viability | Viability |
| 13A | 80% | 100% | 20.30% |
| 13B | 43.90% | | 56.10% |
| 13C | 69.20% | | 30.80% |
| 13D | 69.60% | | 30.40% |
| 13E | 27.00% | | 73.00% |

TABLE 53
MSC surface marker analysis data

| | % Cells expressing the marker | | |
|---|---|---|---|
| Combination # | CD45 | CD90 | CD73 |
| 13A | −0.1% | 93.5% | 88.80% |
| 13B | 1.1% | 93.5% | 90.60% |
| 13C | −1.8% | 90.2% | 90.30% |
| 13D | 0% | 94.6% | 93.80% |
| 13E | −0.1% | 81.5% | 82.10% |

From the above examples, 76 combinations such as 3A, 3B, 3C, 3D, 3E, 3F, 4A, 4B, 4C, 4E, 4F, 4G, 5B, 5C, 5E, 5H, 5I, 5J, 5Q, 5R, 6B, 6C, 7A, 7B, 7C, 7D, 7E, 7F, 7G, 7H, 7I, 7J, 7K, 7L, 7M, 7N, 7O, 7P, 7Q, 7R, 7S, 7T, 7U, 7V, 7W, 8C, 8E, 8H, 8J, 8K, 8L, 8M, 10A, 10B, 11A, 11C, 11D, 11E, 11G, 11H, 11I, 11J, 11K, 11L, 11M, 11N, 11O, 11P, 11Q, 11R, 11 S, 11T, 11U, 12B, 12C, 12E described herein have showed decrease in viability of less than 30% post-lyophilization (viability >70% ).

Summarized below, in table 54, are the comparative post-lyophilization cell viability results, where the lyophilized cells were stored at 2-8° C. and analysed at various time points for post-lyophilization cell viability.

TABLE 54

| | | | | PL % | | PL % | | PL % | | PL % | | PL % | | PL % |
|---|---|---|---|---|---|---|---|---|---|---|---|---|---|---|
| | | | | Cell | | Cell | | Cell | | Cell | | Cell | | Cell |
| | | | | viability | | viability | | viability | | viability | | viability | | viability |
| Components | | | | after 2 | | after 10 | | after 16 | | after 30 | | after 60 | | after 165 |
| Trehalose | H.S.A. | Dextran | CN | days | CN | days | CN | days | CN | days | CN | days | CN | days |
| 500 mM | — | — | 3A | 83.40 | 4A | 73.5 | 6A | 53.30 | — | — | 12A | 65.20 | — | — |
| — | 4% | — | — | — | 4B | 87.9 | — | — | — | — | 12B | 82.00 | — | — |
| — | 5% | — | 3C | 85.80 | — | — | 6B | 83.90 | — | — | 12C | 80.90 | 13A | 80 |
| 150 mM | 5% | — | 3D | 86.40 | 4C | 72 | 6C | 70.80 | — | — | 12D | 67.20 | — | — |
| — | — | 1% | — | — | 4E | 86.8 | — | — | 10A | 72.90 | 12E | 68.90 | 13B | 43.9 |
| 150 mM | — | 8% | 3E | 81.50 | — | — | — | — | 10B | 77.00 | — | — | 13C | 69.2 |
| 500 mM | — | 8% | 3F | 69.70 | — | — | — | — | — | — | 12F | 65.30 | — | — |

CN = Combination Number; PL = post-lyophilization

The result from above table indicates that various combinations have shown reproducible cell viability and are even stable after storage for longer days (2 to 165 days).

1. A lyophilized powder of mesenchymal stem cells.

2. The mesenchymal stein cells lyophilized powder according to claim 1, wherein the mesenchymal stem cells are selected from the group consisting of umbilical cord mesenchymal stein cells, placental mesenchymal stein cells, adipose tissue derived mesenchymal stem cells, limbal tissue derived mesenchymal stem cells and bone marrow mesenchymal stem cells, and its combination.

3. The mesenchymal stem cells lyophilized powder according to claim 1, wherein the mesenchymal stem cells are adipose tissue derived mesenchymal stem cells.

4. The mesenchymal stein cells lyophilized powder according to claim 1, wherein the mesenchymal stem cells are human mesenchymal stem cells.

5. The mesenchymal stem cells lyophilized powder according to claim 1, wherein the mesenchymal stem cells are human adipose tissue derived mesenchymal stem cells.

6. The mesenchymal stem cells lyophilized powder according to any one of claims 1 to 5, wherein the mesenchymal stem cells are exposed to different lyophilisation protocols in presence of various combinations of ingredients.

7. The mesenchymal stem cells lyophilized powder according to any one of claims 1 to 5, wherein the mesenchymal stein cells in a lyophilisation mixture comprises various combinations of ingredients.

8. The mesenchymal stem cells lyophilized powder comprising ingredients according to claim 7, wherein the ingredients are selected from one or more from lyoprotectants, human serum albumin, Glycerol, polyethylene glycol (PEG).

9. The mesenchymal stem cells lyophilized powder comprising ingredients according to claim 7, wherein the ingredient is human serum albumin.

10. The mesenchymal stem cells lyophilized powder comprising ingredients according to claim 8, wherein the lyoprotectants are selected from one or a mixture of several of trehalose, sucrose, lactose, glucose, raffinose, dextran, mannitol, sorbitol or xylitol.

11. The mesenchymal stem cells lyophilized powder comprising ingredients according to claim 8 and claim 10, wherein the lyoprotectant is trehalose.

12. The mesenchymal stem cells lyophilized powder comprising ingredients according to claim 8 and claim 10, wherein the lyoprotectant is dextran.

13. The mesenchymal stem cells lyophilized powder comprising ingredients according to claim 8 and claim 10, wherein the lyoprotectants are combination of trehalose and dextran.

14. The mesenchymal stem cells lyophilized powder according to any one of claims 1 to 13, wherein the mesenchymal stem cells in a lyophilisation mixture comprises human serum albumin.

15. The mesenchymal stein cells lyophilized powder according to any one of claims 1 to 13, wherein the mesenchymal stem cells in a lyophilisation mixture comprises: (a) human serum albumin, and (b) trehalose.

16. The mesenchymal stem cells lyophilized powder according to any one of claims 1 to 13, wherein the mesenchymal stein cells in a lyophilisation mixture comprises: (a) human serum albumin, (b) trehalose, and (c) Glycerol.

17. The mesenchymal stem cells lyophilized powder according to any one of claims 1 to 13, wherein the mesenchymal stem cells in a lyophilisation mixture comprises: (a) human serum albumin, (b) trehalose, (c) Glycerol, and (d) Dextran.

18. The mesenchymal stein cells lyophilized powder according to any one of claims 1 to 13, wherein the mesenchymal stem cells in a lyophilisation mixture comprises: (a) human serum albumin, (b) trehalose, (c) Glycerol, (d) Dextran, and (e) polyethylene glycol (PEG).

19. The mesenchymal stem cells lyophilized powder according to any one of claims 1 to 13, wherein the mesenchymal stem cells in a lyophilisation mixture comprises: (a) human serum albumin, (b) trehalose, (c) Glycerol, (d) Dextran, and (e) PEG 400.

20. The mesenchymal stein cells lyophilized powder according to any one of claims 1 to 19, wherein the mesenchymal stem cells viability post lyophilisation is between about 15% to about 97%.

21. The mesenchymal stem cells lyophilized powder according to any one of claims 1 to 19, wherein the mesenchymal stem cells viability post lyophilisation is between about 25% to about 90%.

22. The mesenchymal stem cells lyophilized powder according to any one of claims 1 to 20, wherein the mesenchymal stem cells viability post lyophilisation is reduced or declined to about 0% to about 30%.

23. The mesenchymal stem cells according to any one of claims 1 to 22, wherein the lyophilized mesenchymal stem cells form a pharmaceutically acceptable cake after lyophilisation.

24. The mesenchymal stem cells lyophilized powder according to any one of claims 1 to 23, wherein the lyophilized mesenchymal stem cells is advantageous for Long-term preservation, easy transportation and distribution of samples in a cost effective way.

25. The mesenchymal stem cells according to any one of claims 1 to 24 are stored at room temperature.

26. The mesenchymal stem cells according to any one of claims 1 to 24 are safe and easy for transportation.

27. The mesenchymal stem cells according to any one of claims 1 to 24 are stable after transportation.

28-37. (canceled)
